(19) United States
(12) Patent Application Publication
(10) Pub. No.: US 2012/0129818 A1
(43) Pub. Date: May 24, 2012

(54) Compositions and Methods for Treatment of Cardiovascular Disease

(76) Inventor: Desikan Rajagopal, Tucker, GA (US)

(21) Appl. No.: 13/265,631

(22) PCT Filed: Apr. 23, 2010
(86) PCT No.: PCT/US2010/032223

§ 371 Date: Feb. 3, 2012

Related U.S. Application Data
(60) Provisional application No. 61/214,425, filed on Apr. 23, 2009.

(51) Int. Cl. A61K 31/635; C07D 319/18; C07D 317/72; C07C 69/773; C07D 405/12; A61K 31/36; A61K 31/357; A61K 31/235; A61K 31/4155; C07D 207/16; A61K 31/40; A61K 31/4709; C07D 211/60; C07D 217/26; C12N 9/99; A61P 9/00; A61P 9/12; A61P 9/10; A61P 29/00; A61P 7/02; A61P 39/06; A61P 9/08; C07D 317/64

(52) U.S. Cl. 514/158; 549/438; 549/410; 549/336; 560/75; 549/404; 549/405; 549/436; 546/283.7; 548/364.4; 514/464; 514/452; 514/462; 514/532; 514/404; 548/533; 546/171; 514/423; 514/314; 546/227; 546/147; 435/184

(57) ABSTRACT

Ortho methoxy phenolic compounds are provided that include methylenedioxyphenyl ferulate and ferulylproline and derivatives thereof. Pharmaceutical compositions comprising the compounds and methods of using the compounds for treating cardiovascular diseases, including hypertension, atherosclerosis, coronary heart disease, angina, stroke, and myocardial infarction, are further provided. The compounds are also useful in reducing low-density lipoprotein oxidation, improving or increasing vasodilation, and reducing plaque destabilization in a subject.

### CROSS-REFERENCE TO RELATED APPLICATIONS

The present application claims the benefit of U.S. Provisional Application Ser. No. 61/214,425, filed Apr. 23, 2009, the entire disclosure of which is incorporated herein by reference.

### FIELD OF THE INVENTION

The present invention relates to compounds and methods for treating cardiovascular diseases. In particular, the present invention relates to ortho methoxy phenolic compounds, including methylenedioxyphenyl ferulate and ferulylproline, and derivatives thereof that can be used to reduce myeloperoxidase (MPO) enzyme activity and thus be useful in the treatment of cardiovascular diseases such as hypertension, atherosclerosis, coronary heart disease, angina, stroke, and myocardial infarction. In addition, the present invention relates to the use of ortho methoxy phenolic compounds, and derivatives thereof, in reducing low- and high-density lipoprotein oxidation, increasing or improving vasodilation, reducing plaque destabilization, reducing the pro-inflammatory nature of high-density lipoproteins, and promoting reverse cholesterol transport in subjects in need of such treatment.

### BACKGROUND OF THE INVENTION

In the United States and other countries, hypertension, stroke, and other diseases related to the cardiovascular system are a major cause of widespread morbidity and mortality, causing great hardship and economic loss to millions of people throughout the world. For example, it has been estimated that nearly 600 million people worldwide are affected with hypertension, with nearly 50 million of those individuals residing in the United States. Furthermore, it has also been estimated that hypertension alone resulted in an annual expenditure of $66.4 billion dollars in the United States alone in 2007.

Despite the widespread hardship and economic consequences associated with hypertension and other cardiovascular diseases, adequate and appropriate treatment of these diseases has still remained elusive for many individuals. The etiology of cardiovascular disease is often multi-factorial and includes a variety of causes such as sedentary lifestyles, obesity, salt sensitivities, alcohol intake, vitamin D deficiency, genetic mutations, and family history. Additionally, recent evidence has indicated that there is a relationship between the development and pathology of cardiovascular disease and myeloperoxidase (MPO) enzyme activity.

MPO is a dimeric enzyme found predominantly in azeotrophic granules of neutrophils and in the lysosomes of monocytes, with monocytes holding only about a third of the MPO that is present in neutrophils. Additionally, promyelocytes and promyelomonocytes vigorously synthesize MPO during granulocyte segregation in the bone marrow. Regardless of the source of MPO, however, in vivo, MPO catalyzes a reaction between hydrogen peroxide (H₂O₂) and chloride anions (Cl), which results in the formation of hypochlorous acid (HOCl), a powerful chlorinating oxidant that reacts with a variety of cellular substrates including heme proteins, porphyrins, thiols, iron sulfur centers, nucleotides, DNA, unsaturated lipids, amines and amino acids. MPO is also believed to oxidize numerous organic and inorganic substrates including aromatic amino acids, indole derivatives and a variety of other species.

The biochemical properties of MPO that contribute to its catalytic effects have been investigated. Indeed, MPO is commonly regarded as providing an important defense mechanism against microorganisms and other infectious pathogens. Despite the advantageous infection-fighting properties of MPO, however, uncontrolled or unwanted MPO activity produces a number of harmful oxidants such as superoxide, hydrogen peroxide, hypochlorous acid, and peroxynitrile, which can harm normal tissue and lead to the development of a number of disease conditions. For example, MPO-catalyzed reactions have been found to exhibit pro-atherogenic biological activity during the development of cardiovascular disease. Furthermore, it has been observed that MPO-generated oxidants reduce the bioavailability of nitric oxide, an important vasodilator. Additionally, it has been shown that MPO plays a role in plaque destabilization, which leads to plaque rupture.

Nevertheless, although MPO has been implicated extensively in the etiology and progression of cardiovascular disease, a biologically safe and non-toxic inhibitor of MPO has yet to be developed. Specifically, an MPO inhibitor has yet to be developed that is based on naturally-occurring bioactive molecules, such that the inhibitor is relatively non-toxic or exhibits only minimally toxicity, but yet is capable of sufficiently inhibiting MPO. It has thus far been difficult to obtain such a suitable compound, and the development of a compound that effectively inhibited MPO would be highly desirable and potentially very beneficial in treating cardiovascular disease.

### SUMMARY OF THE INVENTION

It is thus an object of the present invention to provide compounds and methods for treating cardiovascular disease that can effectively be utilized with minimal toxicity, but yet still sufficiently inhibit myeloperoxidase (MPO) enzyme activity and derive the benefits therein.

It is also an object of the present invention to provide methods of reducing MPO enzyme activity wherein MPO is contacted with an effective amount of a compound of the present invention.

It is a further object of the present invention to provide methods for treating cardiovascular diseases, including hypertension, atherosclerosis, coronary heart disease, angina, stroke, and myocardial infarction, by administering an effective amount of a compound of the present invention to a subject in need of treatment.

It is another object of the present invention to provide methods for reducing low- and high-density lipoprotein oxidation wherein a subject in need of such a reduction is administered an effective amount of a compound of the present invention to thereby reduce a level of low- or high-density lipoprotein oxidation.

It is yet another object of the present invention to provide a method for improving vasodilation wherein a subject in need of such treatment is administered an effective amount of a compound of the present invention to thereby improve vasodilation.

It is still a further object of the present invention to provide a method for reducing plaque destabilization in a subject in need thereof by administering an effective amount of a compound of the present invention to thereby stabilize a plaque lining the arterial wall of a subject.

These and other objects are provided by virtue of the present invention which comprises ortho methoxy phenolic compounds, such as methylenedioxyphenyl ferulate and ferulylproline, and derivatives thereof that are capable of inhibiting MPO and mediating a variety of therapeutic effects. In a preferred embodiment of the present invention, compounds are provided having the following general Formula (I), or a pharmaceutically-acceptable salt or solvate thereof, as follows:

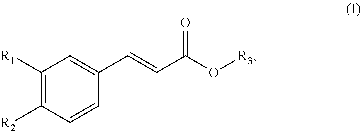

wherein:

R₁ is selected from the group consisting of OCH₃, OH, and OCH₂CH₃;

R₂ is selected from the group consisting of OH and OCH₃; and

R₃ is selected from the group consisting of:

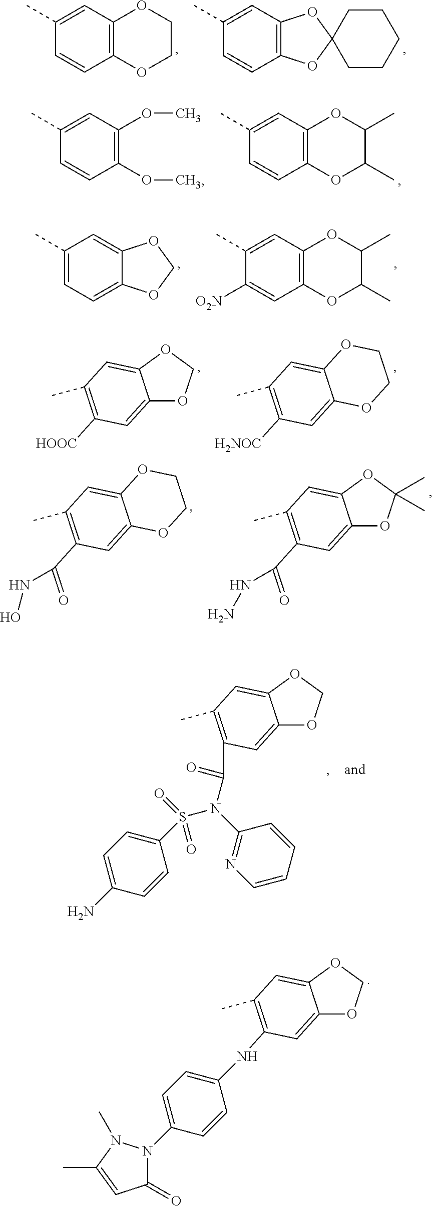

In another preferred embodiment of the present invention, compounds are provided having the general Formula (XV), or a pharmaceutically-acceptable salt or solvate thereof, as follows:

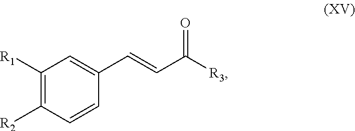

wherein:

R₁ is selected from the group consisting of OCH₃, OCH₂CH₃, and CONHNH₂;

R₂ is selected from the group consisting of OH and NH₂; and

R₃ is selected from the group consisting of:

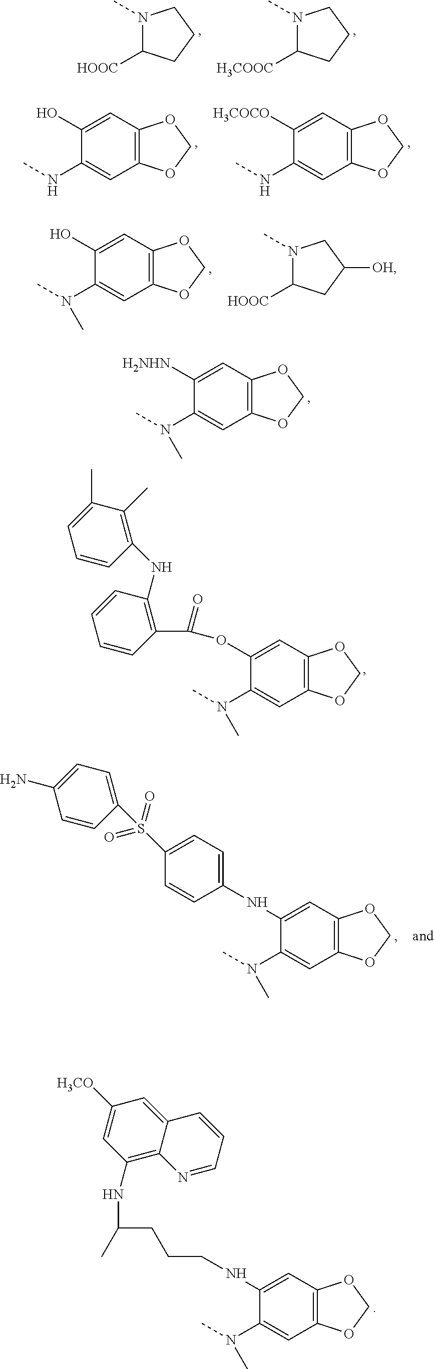

In yet another preferred embodiment of the present invention, compounds are provided having the general Formula (XXVII), or a pharmaceutically-acceptable salt or solvate thereof, as follows:

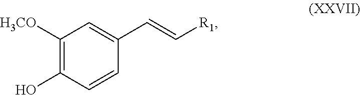

wherein R₁ is selected from the group consisting of:

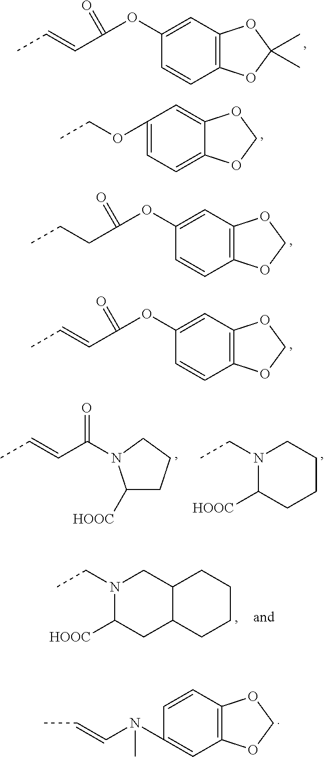

In addition, the present invention provides pharmaceutical compositions wherein the compounds of the present invention further comprise a pharmaceutically-acceptable vehicle, carrier, or excipient.

These embodiments and other alternatives and modifications within the spirit and scope of the presently-disclosed invention will become readily apparent to those of ordinary skill in the art after a study of the description, Figures, and non-limiting Examples in this document.

### BRIEF DESCRIPTION OF THE DRAWING FIGURES

FIG. 1 is a schematic representation showing the chemical coupling reaction utilized to synthesize methylenedioxyphenyl ferulate from ferulic acid and methylenedioxyphenol;

FIG. 2 is a schematic representation showing the chemical coupling reaction utilized to synthesize ferulylproline methyl ester from ferulic acid and proline methyl ester;

FIG. 3 is a schematic representation showing a base-catalyzed hydrolysis reaction utilized to synthesize ferulylproline from ferulylproline methyl ester;

FIG. 4 is a graph showing the inhibition of myeloperoxidase activity by various concentrations of methylenedioxyphenyl ferulate using tetramethylbenzidine as a substrate;

FIG. 5 is a graph showing the inhibition of myeloperoxidase activity by various concentrations of ferulylproline using tetramethylbenzidine as a substrate;

FIG. 6 is a graph showing the effect of ferulylproline and methylenedioxyphenyl ferulate (FMDP) on human low-density lipoprotein (LDL) oxidation;

FIG. 7 is a graph showing the effect of ferulylproline on the inhibition of superoxide mediated cytochrome c reduction catalyzed by xanthine/xanthine oxidase;

FIG. 8 is a graph showing the effect of methylenedioxyphenyl ferulate on the chlorination of taurine and showing methylenedioxyphenyl ferulate inhibition of hypochlorous acid (HOCl)-mediated oxidation;

FIG. 9 is a graph showing the effect of methylenedioxyphenyl ferulate on the formation of a colored product in the presence and in the absence of MPO and showing that methylenedioxyphenyl ferulate itself does not form a product and mask the activity against its substrate;

FIG. 10 is a graph showing the effect of ferulylproline on the formation of a colored product in the presence and in the absence of MPO and showing that ferulylproline itself does not form a product and mask the activity against its substrate.

### DETAILED DESCRIPTION OF THE PREFERRED EMBODIMENTS

In accordance with the present invention, compounds and methods for treating a cardiovascular disease are provided. In particular, the present invention provides ortho methoxy phenolic compounds and derivatives thereof that are capable of sufficiently inhibiting or otherwise reducing myeloperoxidase (MPO) enzyme activity. These compounds are useful in treating a variety of cardiovascular diseases, including hypertension, atherosclerosis, coronary heart disease, angina, stroke, and myocardial infarction. In some embodiments, the compounds can be administered to a subject to reduce low-density lipoprotein oxidation, improve vasodilation, or reduce plaque destabilization in a subject in need of such treatment.

In one of the preferred embodiments of the present invention, compounds useful in the invention have the general Formula (I) as follows:

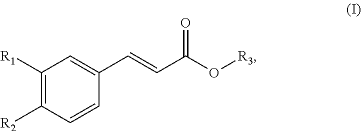

wherein:

R₁ is selected from the group consisting of OCH₃, OH, and OCH₂CH₃;

R₂ is selected from the group consisting of OH and OCH₃; and

R₃ is selected from the group consisting of:

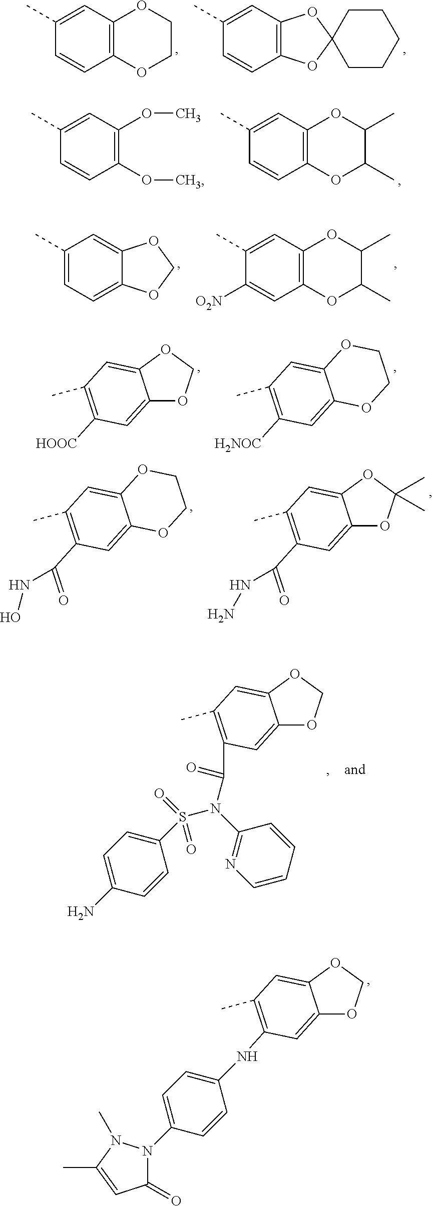

where the dashed bonds (- - -) indicate the point of attachment of the R₃ group to the remainder of the compound.

In one preferred embodiment of the invention, a compound of Formula (I) is provided where R₁ is OCH₃, R₂ is OH, and R₃ is

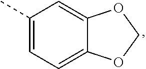

as shown by the following Formula (II):

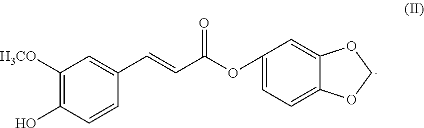

In another preferred embodiment of the invention, a compound of Formula (I) is provided where R₁ is OCH₃, R₂ is OH, and R₃ is

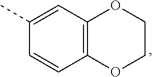

as shown by the following Formula (III):

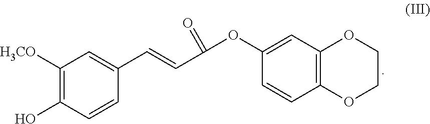

In yet another preferred embodiment of the invention, a compound of Formula (I) is provided where R₁ is OCH₃, R₂ is OH, and R₃ is

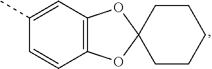

as shown by the following Formula (IV):

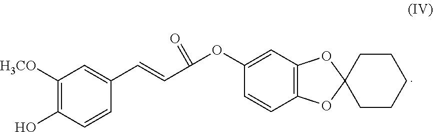

In still another preferred embodiment of the invention, a compound of Formula (I) is provided where R₁ is OCH₃, R₂ is OH, and R₃ is

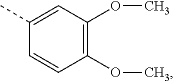

as shown by the following Formula (V):

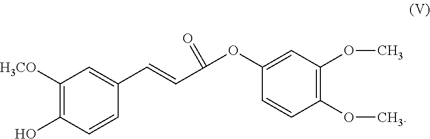

In other preferred embodiments of the invention, a compound of Formula (I) is provided where R₁ is OCH₃, R₂ is OH, and R₃ is

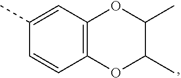

as shown by the following Formula (VI):

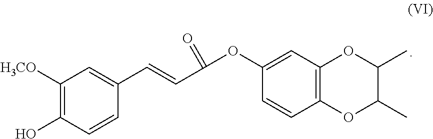

In another embodiment of the invention, a compound of Formula (I) is provided where R₁ is OH, R₂ is OCH₃, and R₃ is

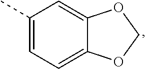

as shown by the following Formula (VII):

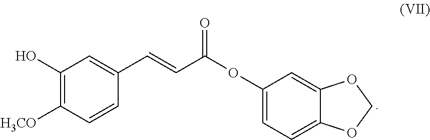

In another embodiment of the invention, a compound of Formula (I) is provided where R₁ is OCH₂CH₃, R₂ is OH, and R₃ is

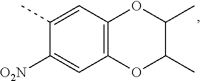

as shown by the following Formula (VIII):

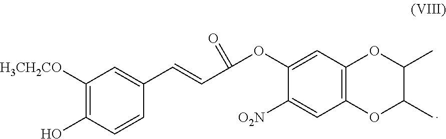

In other embodiments of the invention, a compound of Formula (I) is provided where R₁ is OCH₃, R₂ is OH, and R₃ is

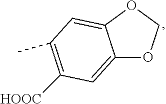

as shown by the following Formula (IX):

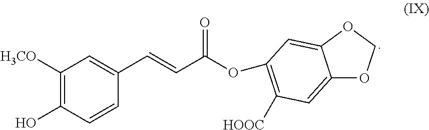

In another preferred embodiment of the invention, a compound of Formula (I) is provided where R₁ is OCH₃, R₂ is OH, and R₃ is

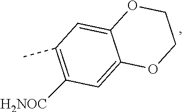

as shown by the following Formula (X):

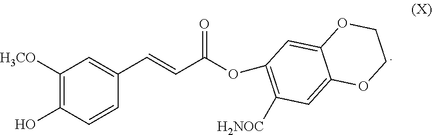

In yet another preferred embodiment of the invention, a compound of Formula (I) is provided where R₁ is OCH₃, R₂ is OH, and R₃ is

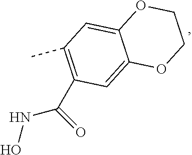

as shown by the following Formula (XI):

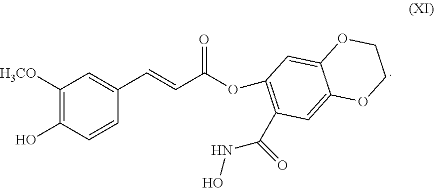

In still another preferred embodiment of the invention, a compound of Formula (I) is provided where R₁ is OCH₃, R₂ is OH, and R₃ is

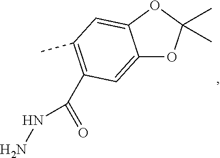

as shown by the following Formula (XII):

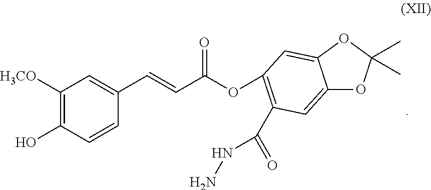

In other preferred embodiments of the invention, a compound of Formula (I) is provided where R₁ is OCH₃, R₂ is OH, and R₃ is

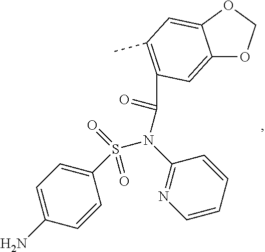

as shown by the following Formula (XIII):

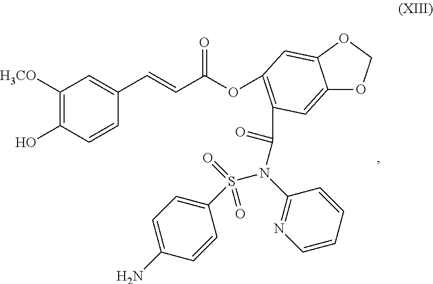

In another preferred embodiment of the invention, a compound of Formula (I) is provided where R₁ is OCH₃, R₂ is OH, and R₃ is

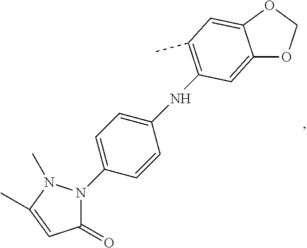

as shown by the following Formula (XIV):

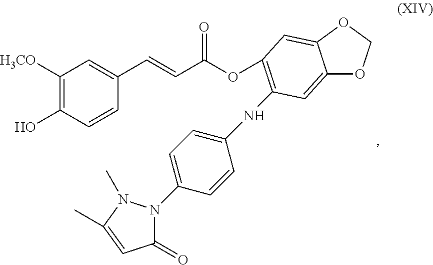

In other embodiments of the present invention, compounds useful in the invention have a general Formula (XV) as follows:

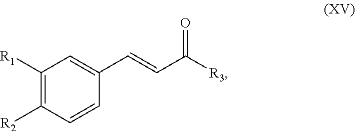

wherein:

R₁ is selected from the group consisting of OCH₃, OCH₂CH₃, and CONHNH₂;

R₂ is selected from the group consisting of OH and NH₂; and

R₃ is selected from the group consisting of:

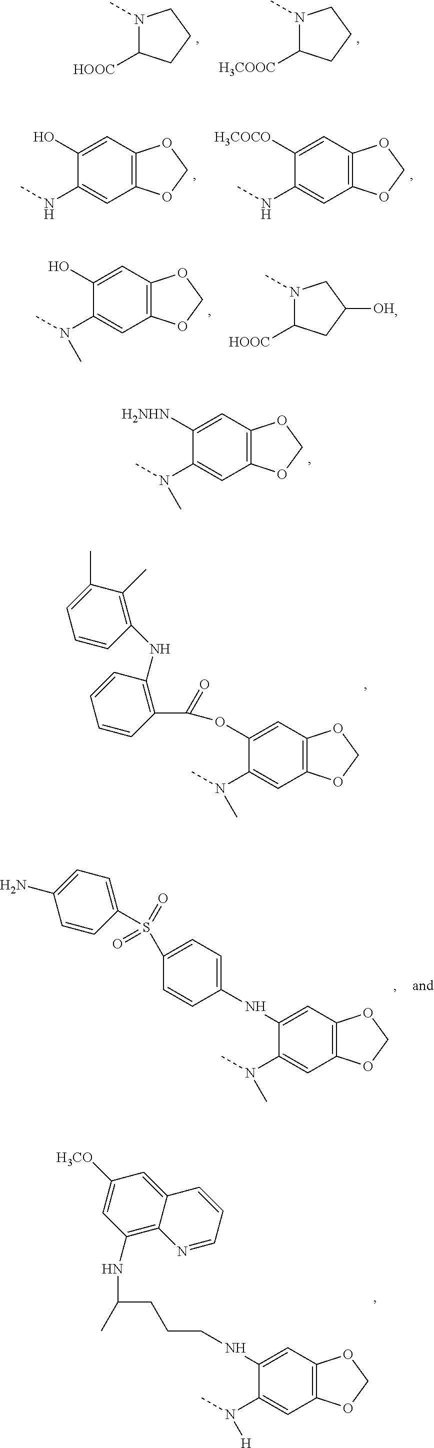

where the dashed bonds (- - -) indicate the point of attachment of the R₃ group to the remainder of the compound.

In one preferred embodiment of the invention, a compound of Formula (XV) is provided where R₁ is OCH₃, R₂ is OH, and R₃ is

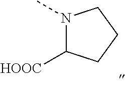

as shown by the following Formula (XVI):

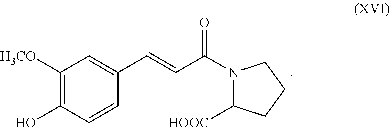

In another preferred embodiment of the invention, a compound of Formula (XV) is provided where R₁ is OCH₃, R₂ is OH, and R₃ is

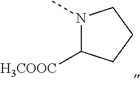

as shown by the following Formula (XVII):

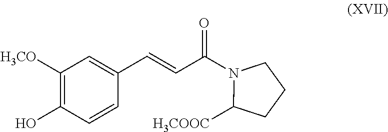

In yet another preferred embodiment of the invention, a compound of Formula (XV) is provided where R₁ is OCH₃, R₂ is OH, and R₃ is

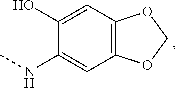

as shown by the following Formula (XVIII):

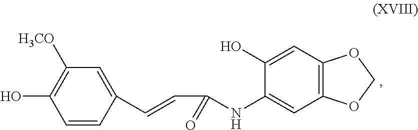

In still another preferred embodiment of the invention, a compound of Formula (XV) is provided where R₁ is OCH₃, R₂ is OH, and R₃ is

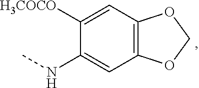

as shown by the following Formula (XIX):

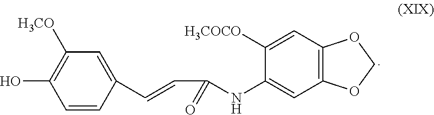

In other preferred embodiments of the invention, a compound of Formula (XV) is provided where R₁ is OCH₂CH₃, R₂ is OH, and R₃ is

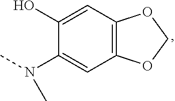

as shown by the following Formula (XX):

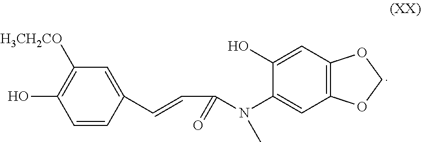

In another embodiment of the invention, a compound of Formula (XV) is provided where R₁ is OCH₃, R₂ is OH, and R₃ is

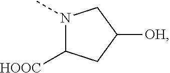

as shown by the following Formula (XXI):

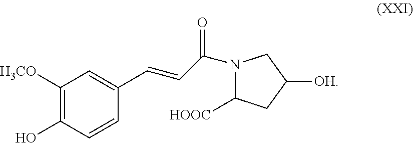

In other embodiments of the invention, a compound of Formula (XV) is provided where R₁ is CONHNH₂, R₂ is NH₂, and R₃ is

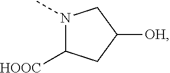

as shown by the following Formula (XXII):

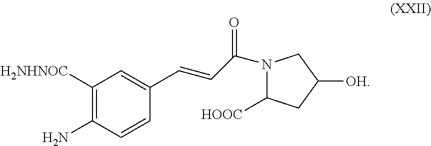

In another preferred embodiment of the invention, a compound of Formula (XV) is provided where R₁ is OCH₂CH₁, R₇ is OH, and R₃ is

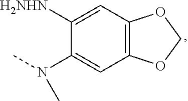

as shown by the following Formula (XXIII):

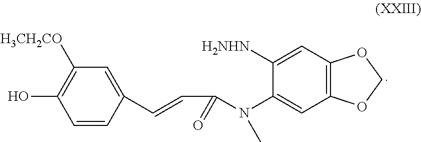

In yet another preferred embodiment of the invention, a compound of Formula (XV) is provided where R₁ is OCH₂CH₃, R₂ is OH, and R₃ is

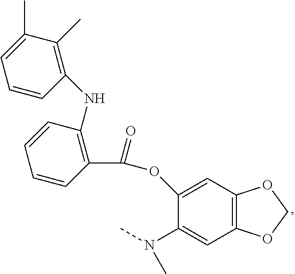

as shown by the following Formula (XXIV):

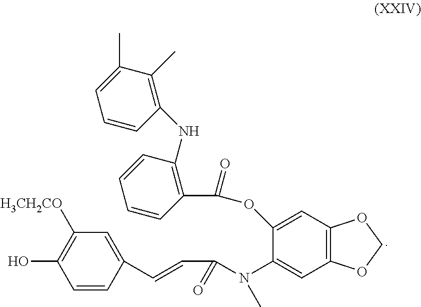

In still another preferred embodiment of the invention, a compound of Formula (XV) is provided where R₁ is OCH₂CH₃, R₂ is OH, and R₃ is

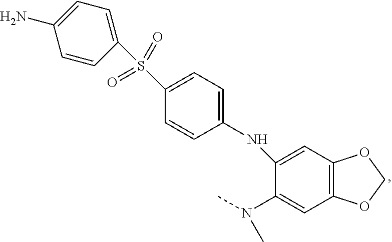

as shown by the following Formula (XXV):

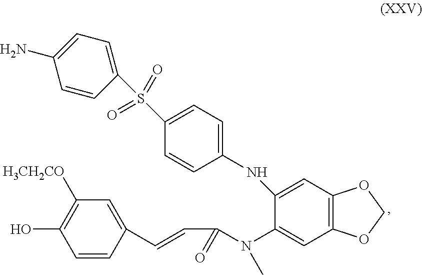

In other preferred embodiments of the invention, a compound of Formula (XV) is provided where R₁ is OCH₃, R₂ is OH, and R₃ is

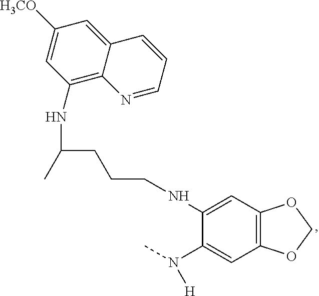

as shown by the following Formula (XXVI):

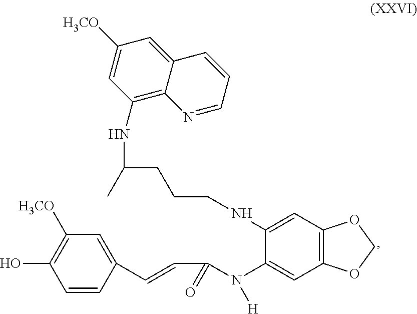

In a further embodiment of the present invention, compounds useful in the invention have the general Formula (XXVII) as follows:

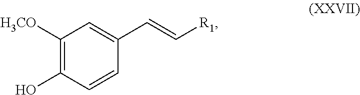

wherein R₁ is selected from the group consisting of:

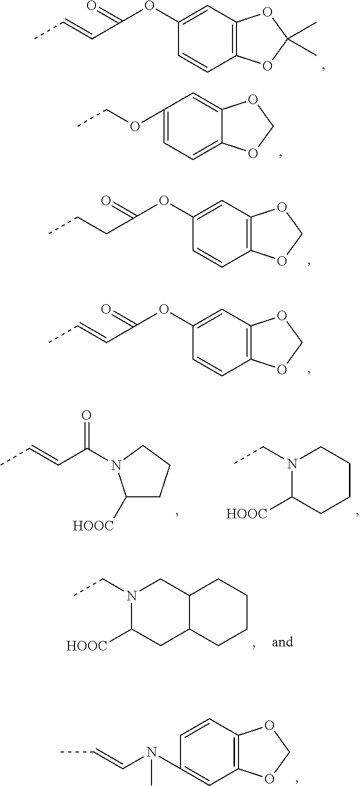

and where the dashed bonds (- - -) indicate the point of attachment of the R₁ group to the remainder of the compound.

In one preferred embodiment of the invention, a compound of Formula (XXVII) is provided where R₁ is

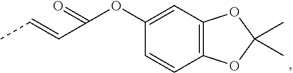

as shown by the following Formula (XXVIII):

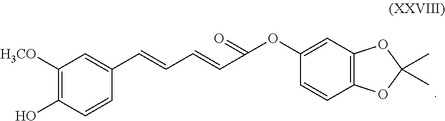

In another preferred embodiment of the invention, a compound of Formula (XXVII) is provided where R₁ is

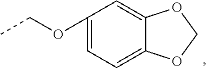

as shown by the following Formula (XXIX):

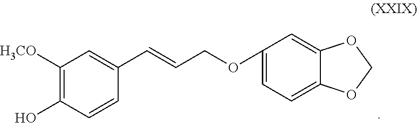

In still another preferred embodiment of the invention, a compound of Formula (XXVII) is provided where R₁ is

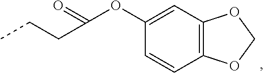

as shown by the following Formula (XXX):

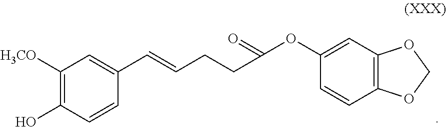

In other preferred embodiments of the invention, a compound of Formula (XXVII) is provided where R₁ is

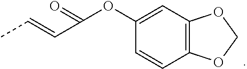

as shown by the following Formula (XXXI):

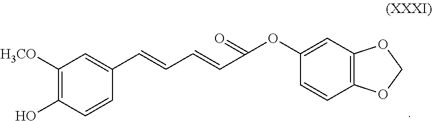

In yet other preferred embodiments of the invention, a compound of Formula (XXVII) is provided where R₁ is

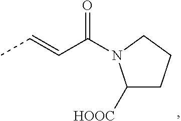

as shown by the following Formula (XXXII):

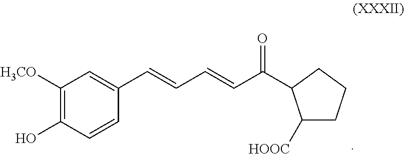

In another preferred embodiment of the invention, a compound of Formula (XXVII) is provided where R₁ is

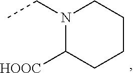

as shown by the following Formula (XXXIII):

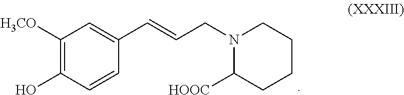

In still another preferred embodiment of the invention, a compound of Formula (XXVII) is provided where R₁ is

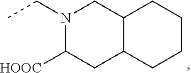

as shown by the following Formula (XXXIV):

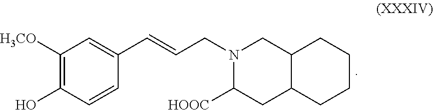

In yet another preferred embodiment of the invention, a compound of Formula (XXVII) is provided where R₁ is

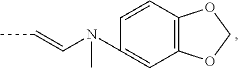

as shown by the following Formula (XXXV):

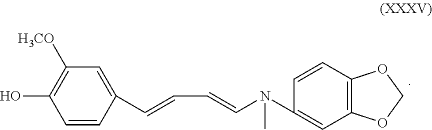

In some embodiments of the present invention, a compound of the present invention is selected from the group consisting of the Formulas (II) and (XVI), shown herein above. In this regard, in some embodiments of the present invention, the compound is selected from methylenedioxyphenyl ferulate (Formula (II)) and ferulylproline (Formula (XVI)). Ferulic acid, or ferulate, is an organic derivative of trans-cinnamic acid with an ortho-methoxy phenol structure. It is an abundant phenolic phytochemical in plant cell walls, and is widely found in the seeds and leaves of most plants, chiefly in the bran of grasses such as wheat, rice and oats. Recently, ferulic acid has been investigated for its potential ability to act as an anti-tumor agent. It has been discovered, however, that, by combining ferulic acid with methylenedioxyphenol or proline, compounds can be developed that effectively inhibit or otherwise reduce the activity of MPO such that these compounds can be used to treat cardiovascular disease. As such, in some embodiments, methylenedioxyphenyl ferulate (Formula (II)) and ferulylproline (Formula (XVI)) compounds are provided that are useful in treating cardiovascular disease. In other embodiments, compounds that are derived from methylenedioxyphenyl ferulate (Formula (II)) and ferulylproline (Formula (XVI)) are provided and are also useful in the treatment of cardiovascular disease.

In addition, and as indicated above, the compounds included herein are described with reference to formulas where one or more additional moieties can be incorporated into the core structure. In these embodiments, reference to the compounds of the present invention can include stereoisomers of the one or more moieties of the compounds. Such stereoisomers are representative of some embodiments of the compounds; however, the formulas and reference to the formulas disclosed herein are intended to encompass all active stereoisomers of the depicted compounds. Furthermore, the compounds of the present invention can, in some embodiments, contain one or more additional asymmetric carbon atoms, and can exist in raecemic and optically active forms. All of these other forms are contemplated to be within the scope of the present invention. As such, the compounds of the present invention can exist in stereoisomeric forms and the products obtained can thus be mixtures of the isomers.

In accordance with the present invention, all of the compounds described herein can be provided in the form of a pharmaceutically-acceptable salt or solvate, as would be recognized by one skilled in the art. A salt can be formed using a suitable acid and/or a suitable base. Suitable acids that are capable of forming salts with the compounds of the present invention include inorganic acids such as trifluoroacetic acid (TFA), hydrochloric acid (HCl), hydrobromic acid, perchloric acid, nitric acid, thiocyanic acid, sulfuric acid, phosphoric acetic acid, propionic acid, glycolic acid, lactic acid, pyruvic acid, oxalic acid, malonic acid, succinic acid, maleic acid, fumaric acid, anthranilic acid, cinnamic acid, naphthalene sulfonic acid, sulfanilic acid, or the like. Suitable bases capable of forming salts with the compounds of the present invention include inorganic bases such as sodium hydroxide, ammonium hydroxide, potassium hydroxide and the like; and organic bases such as mono-, di- and tri-alkyl and aryl amines (e.g., triethylamine, diisopropyl amine, methyl amine, dimethyl amine, and the like), and optionally substituted ethanolamines (e.g., ethanolamine, diethanolamine, and the like).

As used herein, the term “solvate” refers to a complex or aggregate formed by one or more molecules of a solute, e.g., a compound of the present invention or a pharmaceutically-acceptable salt thereof, and one or more molecules of a solvent. Such solvates are typically crystalline solids having a substantially fixed molar ratio of solute and solvent. Representative solvents include, but are not limited to, water, methanol, ethanol, isopropanol, acetic acid, and the like. When the solvent is water, the solvate formed is a hydrate. As such, the term “pharmaceutically-acceptable salt or solvate thereof” is intended to include all permutations of salts and solvates, such as a solvate of a pharmaceutically-acceptable salt of the present compounds.

In yet another embodiment of the compounds of the present invention, and as described further below, pharmaceutical compositions are provided which comprise the compounds described herein and a pharmaceutically acceptable vehicle, carrier or excipient. For example, solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, but are not limited to, microcrystalline cellulose, corn starch, sodium starch glycolate, and alginic acid. Tablet binders that can be used include acacia, methylcellulose, sodium carboxymethylcellulose, polyvinylpyrrolidone (POVIDONE™), hydroxypropyl methylcellulose, sucrose, starch, and ethylcellulose. Lubricants that can be used include magnesium stearates, stearic acid, silicone fluid, talc, waxes, oils, and colloidal silica. Further, the solid formulations can be uncoated or they can be coated by known techniques to delay disintegration and absorption in the gastrointestinal tract and thereby provide a sustained/extended action over a longer period of time. For example, glyceryl monostearate or glyceryl distearate can be employed to provide a sustained-/extended-release formulation. Numerous techniques for formulating sustained release preparations are known to those of ordinary skill in the art and can be used in accordance with the present invention, including the techniques described in the following references: U.S. Pat. Nos. 4,891,223; 6,004,582; 5,397,574; 5,419,917; 5,458,005; 5,458,887; 5,458,888; 5,472,708; 6,106,862; 6,103,263; 6,099,862; 6,099,859; 6,096,340; 6,077,541; 5,916,595; 5,837,379; 5,834,023; 5,885,616; 5,456,921; 5,603,956; 5,512,297; 5,399,362; 5,399,359; 5,399,358; 5,725,883; 5,773,025; 6,110,498; 5,952,004; 5,912,013; 5,897,876; 5,824,638; 5,464,633; 5,422,123; and 4,839,177; and WO 98/47491, each of which is incorporated herein by this reference.

In one preferred embodiment, a sustained-release formulation of a compound of the present invention is provided that utilizes a polyanhydride-based technology. As will be recognized by those skilled in the art, polyanhydrides are a distinctive class of polymers for drug delivery because of their biodegradability and biocompatibility properties. In some embodiments, the release rate of polyanhydride-based formulations can be tuned over several folds by incorporating changes in the polymer structure. As such, in some embodiments of the sustained-release formulations of the presently-described compounds, the polymers employed to provide a sustained-release formulation are selected from poly[1,3-bis(p-carboxyphenoxy)propane, poly[1,3-bis(p-carboxyphenoxy)hexane-co-sebacic anhydride], poly[1,3-bis(p-carboxyphenoxy)methan-co-sebacic anhydride], and poly(fumaric anhydride). Apart from polyanhydride based formulations, in some embodiments, chitosan-based control release technology can be employed to provide a sustained-release formulation, as described further below.

Furthermore, liquid formulations of the compounds for oral administration can be prepared in water or other aqueous vehicles, and can contain various suspending agents such as methylcellulose, alginates, tragacanth, pectin, kelgin, carrageenan, acacia, polyvinylpyrrolidone, and include solutions, emulsions, syrups, and elixirs containing, together with the active components of the composition, wetting agents, sweeteners, and coloring and flavoring agents.

Various liquid and powder formulations can also be prepared by conventional methods for inhalation into the lungs of the subject to be treated. For example, the compositions can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. Capsules and cartridges of, for example, gelatin for use in an inhaler or insufflator may be formulated containing a powder mix of the desired compound and a suitable powder base such as lactose or starch.

Injectable formulations of the compounds can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol), and the like. For intravenous injections, water soluble versions of the compounds can be administered by the drip method, whereby a formulation including a pharmaceutical composition of the present invention and a physiologically-acceptable excipient is infused. Physiologically-acceptable excipients can include, for example, 5% dextrose, 0.9% saline, Ringer's solution or other suitable excipients. Intramuscular preparations, e.g., a sterile formulation of a suitable soluble salt form of the compounds, can be dissolved and administered in a pharmaceutical excipient such as Water-for-Injection, 0.9% saline, or 5% glucose solution. A suitable insoluble form of the compound can be prepared and administered as a suspension in an aqueous base or a pharmaceutically-acceptable oil base, such as an ester of a long chain fatty acid, (e.g., ethyl oleate).

In addition to the formulations described above, the compounds of the present invention can also be formulated as rectal compositions, such as suppositories or retention enemas, e.g., containing conventional suppository bases such as cocoa butter or other glycerides. Further, the compositions can also be formulated as a depot preparation by combining the compositions with suitable polymeric or hydrophobic materials (for example as an emulsion in an acceptable oil) or ion exchange resins, or as sparingly soluble derivatives, for example, as a sparingly soluble salt.

In some embodiments of the present invention, the compounds of the present invention may be incorporated into a nanoparticle. A nanoparticle within the scope of the invention is meant to include particles at the single molecule level as well as those aggregates of particles that exhibit microscopic properties. Methods of using and making a nanoparticle that incorporates a compound of interest are known to those of ordinary skill in the art and can be found following references: U.S. Pat. Nos. 6,395,253, 6,387,329, 6,383,500, 6,361,944, 6,350,515, 6,333,051, 6,323,989, 6,316,029, 6,312,731, 6,306,610, 6,288,040, 6,272,262, 6,268,222, 6,265,546, 6,262,129, 6,262,032, 6,248,724, 6,217,912, 6,217,901, 6,217,864, 6,214,560, 6,187,559, 6,180,415, 6,159,445, 6,149,868, 6,121,005, 6,086,881, 6,007,845, 6,002,817, 5,985,353, 5,981,467, 5,962,566, 5,925,564, 5,904,936, 5,856,435, 5,792,751, 5,789,375, 5,770,580, 5,756,264, 5,705,585, 5,702,727, and 5,686,113, each of which is incorporated herein by this reference.

Nanoparticles are frequently regarded as solid colloidal particles ranging in size from 10 nm to 1 μm, and can be built from macromolecular assemblies, in which an active compound or agent (e.g., a compound of the present invention) is dissolved, entrapped, encapsulated, or adsorbed or attached to the external interface to provide kinetic stability and rigid morphology. In some embodiments of the present invention, a bio-polymer-based nanoparticle formulation is utilized for efficient delivery of a compound of the presently-disclosed subject matter. In some embodiments, a formulation can be provided that utilizes chitosan/polyguluronate nanoparticles, poly(D,L-lactic acid)/ethyl acetate-based nanoparticles, PLGA-, PLGA:poloxamer-, or PLGA:poloxamine/dichloromethane-mediated nanoparticles, PEGylated polymeric micelles, or nanoparticles of albumin. As will be recognized by those of skill in the art, the preparation of nanoparticles as a composition vehicle will depend on the types of biopolymers employed in the process.

In one preferred embodiment of the present invention, a nanoparticle formulation can be provided that is derived from a chitosan/polyguluronate combination. Chitosan is a naturally existing polysaccharide composed of glucosamine and N-acetylglucosamine residues and can be derived by partial deacetylation of chitin, which is generally obtained from crustacean shells. Chitosan is known to be a biocompatible, low toxic, low immunogenic, and degradable by enzymes. In this regard, a nanoparticle formulation of the compounds of the present invention can be prepared by first dissolving chitosan glutamate in a suitable buffer, and, similarly, dissolving polyguluronate in a sodium sulfate buffer. The solutions can then be filtered through a micro-filter, and the nanoparticle formulations can then be prepared by adding the chitosan solution to an equal volume of the polyguluronate solution and then incubating the particles room temperature. In this regard, to incorporate a compound of the present invention into the nanoparticles, a desired amount of the compound, in a polar solvent, can be first added to the polyguluronate solution, and then the mixture can be combined with the chitosan solution. The resulting nanoparticles can then be incubated at room temperature before use or further analysis (see, e.g., Hoffman A S, The origins and evolution of “controlled” drug delivery systems, Journal of Controlled Release, 132 (2008), 153-163).

With further regard to the compounds of the present invention, it is again noted that the compounds of the present invention include derivatives of ortho methoxy phenolic compounds, such as derivatives of methylenedioxyphenyl ferulate and derivatives of ferulylproline. As used herein, the term “derivative” refers to a chemically or biologically modified version of a chemical compound that is structurally similar to the parent compound and derivable from that parent compound. A “derivative” differs from an “analogue” in that a parent compound can be the starting material to generate a “derivative,” whereas the parent compound may not necessarily be used as the starting material to generate an “analogue.”Additionally, a derivative may or may not have different chemical or physical properties of the parent compound. For example, the derivative may be more hydrophilic or it may have altered reactivity as compared to the parent compound. In this regard, derivatization (i.e., modification) may involve substitution of one or more moieties within the molecule (e.g., a change in functional group). For example, a hydrogen may be substituted with a halogen, such as fluorine or chlorine, or, as another example, a hydroxyl group (—OH) may be replaced with a carboxylic acid moiety (—COON).

As used herein, the term “derivative” also includes conjugates and prodrugs (i.e., chemically modified derivatives which can be converted into the original compound under physiological conditions) of a parent compound. For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). Further information relating to prodrugs is found, for example, in Fleisher et al., Advanced Drug Delivery Reviews 19 (1996) 115; Design of Prodrugs, H. Bundgaard (ed.), Elsevier, 1985; or H. Bundgaard, Drugs of the Future 16 (1991) 443, each of which is incorporated herein by this reference.

In accordance with the present invention, methods are further provided for reducing myeloperoxidase (MPO) enzyme activity by contacting MPO with an effective amount of a compound selected from the compounds of Formulas (I), (XV), and (XXVII) or pharmaceutically-acceptable salts or solvates thereof. As noted herein, MPO is an enzyme found predominantly in azeotrophic granules of neutrophils and in the lysosomes of monocytes. In vivo, MPO produces hypochlorous acid (HOCl), from hydrogen peroxide (H₂O₂) and a chloride anion (Cl⁻), which, once produced, is a powerful chlorinating oxidant capable of reacting with a variety of cellular substrates. Furthermore, MPO itself is capable of oxidizing certain amino acids on proteins by using hydrogen peroxide as an oxidizing agent. As such, “reducing” or a “reduction of” of MPO activity includes, but is not limited to, a reduction in HOCl formation and a reduction in protein oxidation. Further, it is understood that the degree of reduction need not be absolute (e.g., complete inhibition of MPO activity such that no HOCl is formed), and that intermediate levels of reduction are contemplated by the present invention including, but not limited to, reductions of about 5%, about 10%, about 15%, about 20%, about 30%, about 40%, about 50%, about 60%, about 70%, about 75%, about 80%, about 85%, about 90%, about 95%, and about 99% reductions in the amount of MPO activity.

Various methods of measuring a reduction in MPO activity are known to those of ordinary skill in the art and can be used in accordance with the present invention. For example, in one embodiment, MPO activity can be measured by combining an amount of an MPO enzyme, or a sample containing an MPO enzyme, with H₂O₂, a tetramethyl benzidine (TMB) substrate, and an MPO inhibitor (e.g., a compound of the present invention), and then measuring the optical density of the resulting product.

As will also be recognized by those of ordinary skill in the art, in embodiments where contacting an MPO enzyme with a compound of Formulas (I), (XV), or (XXVII) reduces MPO activity, the optimum amount of the compound used to reduce MPO activity can vary depending on the desired degree of reduction. In certain embodiments, MPO activity is reduced by contacting MPO with a concentration of the compound in the range of about 1 to about 25 μM, such as a concentration of about 5 μM. In other embodiments, MPO activity is reduced by contacting MPO with an effective amount of the compound by administering to a subject a dose of the compound of about 10 to about 800 mg per day. As disclosed herein, data and, specifically, Kᵢ values that were generated by contacting MPO with methylenedioxyphenyl ferulate (Formula (II)) and ferulylproline (Formula (XVI)) indicated that MPO activity can effectively be inhibited using concentrations of the two compounds in the range of 1 to 10 μM. Without wishing to be bound by any particular theory, however, it is contemplated that these Kᵢ values can further be reduced by appropriate chemical modification to nanomolar concentrations, such as between about 50 nM and about 100 nM. Of course, determination and adjustment of the effective amount of a compound of the present invention to be used in a particular application, as well as when and how to make such adjustments, would be readily ascertainable by those of ordinary skill in the art using only routine experimentation or other routine techniques.

In accordance with the present invention, methods for treating a cardiovascular disease are also provided. In one preferred embodiment, a method for treating a cardiovascular disease is provided that comprises administering to a subject an effective amount of a compound selected from Formulas (I), (XV), or (XXVII) of the present invention, or pharmaceutically acceptable salts or solvates thereof, to thereby treat the cardiovascular disease in the subject.

As used herein, the terms “treatment” or “treating” relate to any treatment of a cardiovascular disease, including but not limited to prophylactic treatment and therapeutic treatment. As such, the terms “treatment” or “treating” include, but are not limited to: preventing a cardiovascular disease or the development of a cardiovascular disease; inhibiting the progression of a cardiovascular disease; arresting or preventing the further development of a cardiovascular disease; reducing the severity of a cardiovascular disease; ameliorating or relieving symptoms associated with a cardiovascular disease; and causing a regression of a cardiovascular disease or one or more of the symptoms associated with a cardiovascular disease.

The term “cardiovascular disease” is used herein to refer to any disease or disorder that affects, at least in part, the cardiovascular system including the heart and blood vessels (e.g., arteries and veins) of a subject. As noted herein, MPO has been found to exhibit pro-atherogenic biological activity during the evolution of cardiovascular disease. Furthermore, it has been observed that MPO-generated oxidants reduce the bioavailability of nitric oxide, an important vasodilator. Additionally, is has been shown that MPO plays a role in plaque destabilization by causing the activation of metalloproteinases, leading to a weakening of the fibrous cap of the plaques and subsequent plaque destabilization and rupture. Given these wide-ranging effects of MPO, MPO has thus been implicated in a wide variety of cardiovascular diseases including, but not limited to, hypertension, atherosclerosis, coronary heart disease, angina, stroke, and myocardial infarction. As such, in certain embodiments, the cardiovascular disease is selected from the group consisting hypertension, atherosclerosis, coronary heart disease, angina, stroke, and myocardial infarction.

In some embodiments of the methods for treating a cardiovascular disease, the compounds of the present invention (i.e., compounds of Formulas (I), (XV), or (XXVII)) can be combined with various other therapeutic agents to provide a pharmaceutical composition that can be effectively used to treat a cardiovascular disease, as defined herein. In some embodiments, a compound of the present invention is administered in combination with an angiotensin-converting enzyme inhibitor, an angiotensin II receptor blocker, a statin, an anti-inflammatory agent, an agent that inhibits the absorption of fatty acids, an anti-platelet agent, an anti-coagulant, or combinations thereof to treat the cardiovascular disease.

For example, in some embodiments of the presently-disclosed methods for treating a cardiovascular disease, a compound of the present invention is combined with an angiotensin-converting enzyme inhibitor and/or an angiotensin II receptor blocker to thereby treat the cardiovascular disease. As would be recognized by those skilled in the art, angiotensin-converting enzyme (ACE) is a peptidylcarboxypeptidase, which catalyzes the cleavage of the histidine-leucine dipeptide at the carboxy-terminus of the inactive decapeptide angiotensin I to form angiotensin II, and is also responsible for the deactivation of bradykinase. Once the dipeptide has been cleaved from the carboxy-terminus of angiotensin I and angiotensin II has been formed, angiotensin II is then able to mediate a variety of responses, by binding to and activating the angiotensin receptors AT₁ and AT₂, which subsequently mediate a variety of physiological responses within the cardiovascular system. As such, agents that are capable of inhibiting the conversion of angiotensin I to angiotensin II, e.g., ACE inhibitors, as well as agents that are capable of blocking the binding of angiotensin II to its receptors and thus reducing the activation of the receptors, e.g., angiotensin II receptor blockers or “ARBs” (which may also be referred to as angiotensin II receptor antagonists, AT₁-receptor antagonists, or sartans) are thus useful in treating a variety of different cardiovascular diseases. Numerous ACE inhibitors and ARBs are known to those of ordinary skill in the art and can be used in accordance with the compositions of the present invention. In some embodiments, the ACE inhibitor is selected from the group consisting of benazepril, captopril, cilazapril, enalapril, enalaprilat, fosinopril, lisinopril, moexipril, perindopril, quinapril, ramipril, trandolapril, and zofenopril. In other embodiments, the ARB is selected from the group consisting of candesartan, eprosartan, irbesartan, telmisartan, valsartan, losartan, and olmesartan.

As another example of a pharmaceutical composition that includes a compound of the present invention and an additional agent that is useful for treating a cardiovascular disease, in certain embodiments of the present invention, a statin can be further combined with a compound of Formulas (I), (XV), or (XXVII) to produce a composition of the present invention. Various statins (i.e., HMG-CoA reductase inhibitors) are known to those of ordinary skill in the art as agents that are capable inhibiting the HMG-CoA reductase enzyme and thus decreasing cholesterol synthesis and increasing synthesis of low-density lipoprotein (LDL) receptors, which then results in an increased clearance of LDLs from the blood stream of a subject. In certain embodiments of the compositions described herein, the statin that is combined with a compound of Formulas (I), (XV), or (XXVII) can be selected from atorvastatin, fluvastatin, lovastatin, mevastatin, pitavastatin, pravastatin, rosuvastatin, and simvastatin. Each of these statins can be combined with a composition of the present invention and be useful in treating a cardiovascular disease.

As yet another example of a pharmaceutical composition that includes a compound of the present invention and an additional agent that is useful for treating a cardiovascular disease, in some embodiments, a lipoic acid compound is further combined with a compound of Formulas (I), (XV), or (XXVII) to produce a composition of the present invention. Alpha lipoic acid, also known as thioctic acid, is a naturally-occurring 8-carbon fatty acid that is synthesized by plants and animals, including humans, and serves several important functions in the body. Alpha lipoic acid contains two sulfur atoms that are normally found in an oxidized, disulphide form, but which can be reduced to form thiols and form dihydrolipoic acid (DHLA). Indeed, the body of an individual routinely converts some alpha lipoic acid to DHLA, and it is believed that DHLA may function as a more powerful antioxidant when compared to alpha lipoic acid. As such, the term “lipoic acid compound,” as used herein, is inclusive of alpha lipoic acid, dihydrolipoic acid, and derivatives thereof.

In certain embodiments of the present compositions, and as yet another example of a pharmaceutical composition that includes a compound of the present invention and an additional agent that is useful for treating a cardiovascular disease, a composition can be provided that includes a compound of the present invention and further includes an anti-inflammatory agent. Examples of anti-inflammatory agents which may be used in accordance with the compositions of the present invention include, but are not limited to, classic non-steroidal anti-inflammatory agents (NSAIDS), such as aspirin, diclofenac, indomethacin, sulindac, ketoprofen, flurbiprofen, ibuprofen, naproxen, piroxicam, tenoxicam, tolmetin, ketorolac, oxaprosin, mefenamic acid, fenoprofen, nambumetone (relafen), acetaminophen, and combinations thereof; COX-2 inhibitors, such as nimesulide, flosulid, celecoxib, rofecoxib, parecoxib sodium, valdecoxib, etoricoxib, etodolac, meloxicam, and combinations thereof; glucocorticoids, such as hydrocortisone, cortisone, prednisone, prednisolone, methylprednisolone, meprednisone, triamcinolone, paramethasone, fluprednisolone, betamethasone, dexamethasone, fludrocortisone, desoxycorticosterone, rapamycin; or others or analogues of these agents or combinations thereof.

In other embodiments of the present compositions, an agent that inhibits the absorption of fatty acids, such as ezetimibe, sulfated polysaccharides, oleayl alcohols, or lecithin, can be further combined with a compound of the present invention (i.e., a compound of Formulas (I), (XV), or (XXVII)). Ezetimibe may also be combined with statins to produce combination drugs such as Vytorin® (MSP Singapore Company, LLC, Whitehouse Station, N.J.). Drugs to reduce the tendency of platelet aggregation (i.e., anti-platelet agents), such as clopidogrel bisulfate, or anti-coagulant drugs, such as heparin, can also be administered. Additionally, agents that have actions directly or indirectly on the cardiovascular system can be administered including, but not limited to, niacin, fibrates such as fenofibrate and gemfibrozil, and thiazolidinediones.

For administration of a therapeutic composition as disclosed herein, conventional methods of extrapolating human dosage based on doses administered to a murine animal model can be carried out using the conversion factor for converting the mouse dosage to human dosage: Dose Human per kg=Dose Mouse per kg×12 (Freireich, et al., (1966) Cancer Chemother Rep. 50:219-244). Drug doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m².

Suitable methods for administering a therapeutic composition in accordance with the methods of the present invention include, but are not limited to, systemic administration, parenteral administration (including intravascular, intramuscular, intraarterial administration), oral delivery, buccal delivery, rectal delivery, subcutaneous administration, intraperitoneal administration, inhalation, intratracheal installation, surgical implantation, transdermal delivery, local injection, and hyper-velocity injection/bombardment. Where applicable, continuous infusion can enhance drug accumulation at a target site (see, e.g., U.S. Pat. No. 6,180,082).

Regardless of the route of administration, the compounds of the present invention are typically administered in amount effective to achieve the desired response. As such, the term “effective amount” is used herein to refer to an amount of the therapeutic composition (e.g., a composition comprising a compound of Formula (I), (XV), or (XXVII), and a pharmaceutically vehicle, carrier, or excipient) sufficient to produce a measurable biological response (e.g., a decrease in blood pressure or a reduction in MPO activity). Actual dosage levels of active ingredients in a therapeutic composition of the present invention can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

In certain embodiments of the methods of the present invention in which the administration of a compound of Formulas (I), (XV), or (XXVII) is indicated, the compounds can be administered twice daily at doses ranging from about 10 mg to about 800 mg, about 100 mg to about 700 mg, about 200 mg to about 600 mg or about 300 mg to about 500 mg. In another embodiment, the compounds can be administered once daily at doses ranging from about 10 mg to about 800 mg, about 100 mg to about 700 mg, about 200 mg to about 600 mg, or about 300 mg to about 500 mg.

In certain embodiments of the therapeutic methods described herein where a compound of the present invention in administered in combination with an ACE inhibitor, an ARB, a lipoic acid compound, and or a statin, the ACE inhibitor, the ARB, the lipoic acid compound, and the statin can be combined in a composition at dosage ranges such as those provided in Table 1 below. When an anti-inflammatory agent, an agent that inhibits absorption of fatty acids, an anti-platelet agent, or an anti-coagulant is included in a composition of the present invention, the dosage ranges of those agents can include the dosage ranges that would typically be employed for those specific agents. Of course, additional variations of the doses described herein can be utilized in a composition of the present invention to achieve the desired biological response, and can be ascertained by those of ordinary skill in the art of medicine using routine experimentation.

TABLE 1
Exemplary Dosage Ranges.

| Active Ingredient | Dosage Range |
|---|---|
| ACE Inhibitor or ARB | 0.1 mg/day to 100 mg/day |
| | 1 mg/day to 80 mg/day |
| | 5 mg/day to 50 mg/day |
| | 5, 10, or 20 mg/day |
| Lipoic Acid Compound | 1 mg/day to 1000 mg/day |
| | 10 mg/day to 600 mg/day |
| | 100 mg/day to 400 mg/day |
| | 300, 400, 500 or 600 mg/day |
| Statin | 1 mg/day to 100 mg/day |
| | 10 mg/day to 80 mg/day |
| | 20 mg/day to 60 mg/day |

For additional guidance regarding formulation and dose, see U.S. Pat. Nos. 5,326,902 and 5,234,933; PCT International Publication No. WO 93/25521; Berkow, et al., (1997) The Merck Manual of Medical Information, Home ed. Merck Research Laboratories, Whitehouse Station, N.J.; Goodman, et al., (2006) Goodman & Gilman's the Pharmacological Basis of Therapeutics, 11th ed. McGraw-Hill Health Professions Division, New York; Ebadi. (1998) CRC Desk Reference of Clinical Pharmacology. CRC Press, Boca Raton, Fla.; Katzung, (2007) Basic & Clinical Pharmacology, 10th ed. Lange Medical Books/McGraw-Hill Medical Pub. Division, New York; Remington, et al., (1990) Remington's Pharmaceutical Sciences, 18th ed. Mack Pub. Co., Easton, Pa.; Speight, et al., (1997) Avery's Drug Treatment: A Guide to the Properties, Choice, Therapeutic Use and Economic Value of Drugs in Disease Management, 4th ed. Adis International, Auckland/Philadelphia; and Duch, et al., (1998) Toxicol. Lett. 100-101:255-263, each of which are incorporated herein by reference.

In yet another embodiment of the therapeutic methods described herein, administering an effective amount of a compound of the present invention to a subject reduces an amount of oxidation of a low- or high-density lipoprotein (LDL or HDL) in the subject. The effective amount of a therapeutic composition administered to a subject in accordance with the present invention to reduce LDL or HDL oxidation will vary depending on the subject's circumstances and the desired result to be achieved, but can readily be determined using routine experimentation.

Current research indicates that an abundance of reactive oxygen species in the vasculature of a subject, such as what may be generated as a result of MPO activation, results in an increased oxidation of proteins such as oxidized LDL (ox-LDL), which then initiates an inflammatory process and causes intimal damage to the arterial wall (See, Parthasarathy, et al. Proc Natl Acad Sci USA. 1987, 84(9), 2995-8). While the mechanisms of this damage are not yet established and may involve the inactivation of nitric oxide (NO) by oxygen-derived free radicals such as superoxide, the inflammatory response seen in these subjects has been observed to affect the gene expression of various inflammatory molecules, such as VCAM and tumor necrosis factor-alpha (TNF-α), which in turn can regulate the inflammatory process and promote foam cell formation (See, e.g., Rajagopalan S, Harrison D G. Circulation 1996; 94:240-243; Henninger D D, et al. Circ Res 1997; 81:274-281; Stannard A K, et al. Atherosclerosis 2001; 154:31-38; and, Libby P, et al. Curr Opin Lipidol 1996; 7:330-335). The reduction in NO levels along with an increase in ox-LDL may function as immunomodulators of the atherosclerotic process (See, e.g., Vergnani L, et al. Circulation 2000; 101:1261-1266.). Disclosed herein, however, are data showing that a compound of the present invention can effectively be used to significantly reduce the amount of LDL oxidation.

Various methods of measuring an amount of LDL or HDL oxidation are known to those of ordinary skill in the art and can be used in accordance with the present invention. For example, an amount of LDL oxidation can be measured by obtaining plasma samples from subjects, isolating the LDLs by ultracentrifugation, and then oxidizing the LDL to ox-LDL using a standard assay involving CuSO₄ (See, e.g., Parthasarathy S, et al. Methods Mol. Biol. 2010. 610, 403-17 and references therein). The lag time of oxidation, which indicates the susceptibility of LDL to oxidize, can then be measured using a spectrophotometer to allow the amounts of LDL oxidation occurring in a subject to be ascertained.

In another embodiment of the present invention, a method of improving vasodilation is provided whereby a subject in need of treatment is administered an amount of a compound in accordance with the invention that is effective to improve vasodilation in the subject. Again, the effective amount of a therapeutic composition administered to a subject in accordance with the present invention to improve vasodilation will vary depending on the subject's circumstances and the desired result to be achieved, but can readily be determined using routine experimentation.

Nitric oxide produced by endothelial nitric oxide synthase (eNOS) is known to those of ordinary skill in the art as a potent vasodilator and is further known to mediate several important endothelial functions. Current research, however, indicates that MPO, by multiple mechanisms can reduce the bioavailability of nitric oxide and, consequently, reduce the amount available for vasodilation. For example, hypochlorous acid can react with nitrogen atoms of the nitric oxide synthase substrate arginine to produce chlorinated arginine species, which are able to effectively inhibit all isoforms of nitric oxide synthase and have been shown to impair endothelium-dependent relaxation of aortic rings. As another example, hypochlorous acid is also a potent inducer of the uncoupling of eNOS, thereby turning eNOS into a superoxide-generating enzyme. Without wishing to be bound by any particular theory, however, it is believed that the compounds of the present invention are capable of sufficiently inhibiting MPO activity such that MPO is unable to deplete nitric oxide in various anatomical locations, such as the vascular wall, thus preventing the events outlined above.

Various methods of measuring the extent of vasodilation in a subject can be used in accordance with the present invention, including a non-invasive flow-mediated dilation technique, which uses high-resolution ultrasound to evaluate endothelial-dependent and endothelial-independent vasodilatation in the brachial artery. Briefly, that test stimulates the endothelium of the brachial artery in the arm to release nitric oxide, which then causes vasodilatation of the artery. The resulting vasodilatation can then be measured and quantified as a marker of endothelial function.

In yet another embodiment of the present invention, a method of reducing plaque destabilization is provided whereby a subject in need of treatment is administered an amount of a compound in accordance with the invention that is effective to reduce plaque destabilization in the subject. Once again, the effective amount of a therapeutic composition administered to a subject in accordance with the present invention to reduce plaque destabilization will vary depending on the subject's circumstances and the desired result to be achieved, but can readily be determined using routine experimentation.

The formation of plaques along arterial walls is a hallmark of atherosclerosis and can lead to the many harmful effects. For example, one significant effect of the development of arterial plaques is that as the plaques increase in size over time or become vulnerable, the plaques themselves become destabilized leading to plaque rupture. In many of these cases, such vulnerable caps include thin fibrous caps with a large and soft lipid pool underlying the cap, which makes the cap prone to destabilization and subsequent rupture. In this regard, MPO can further play a role in plaque destabilization by activating metalloproteinases, which then act to weaken the fibrous caps, leading to adverse cardiovascular consequences such as myocardial infarction. Without wishing to be bound by any particular theory though, it is thought that the administration of a compound of the present invention is able to effectively reduce plaque destabilization by inhibiting MPO and, consequently, indirectly inhibiting the activation of metalloproteinases.

As would be recognized by those of ordinary skill in the art, the stability of plaques depends on both the collagen smooth muscle content in the atherosclerotic lesions. In this regard, it is often found that thrombosed plaques contain a lower content of collagen. Further, over-expression of collagenases also changes the mechanical properties of the plaques by their enzymatic actions, which lead to weaker, disorganized collagen fibers. Typically, smooth muscle cells in plaques generate collagen, and their migration and proliferation to the site of an intimal lesions is often associated with a total increase in collagen, thus imparting stability to the plaque. However, diminishing smooth muscle cells may also predispose a plaque to rupture, as evidenced by observations that plaques associated with unstable angina frequently show smooth muscle cell apoptosis.

There are a variety of techniques that are used by those skilled in the art to measure the stability of a plaque, each of which can be used in accordance with the presently-disclosed subject matter. For example, Polar-Sensitive Optical Coherence Tomography (PSOCT) can be used to measure the stability of a plaque as that technique has been shown to measure birefringence, a material property that is elevated in tissues such as collagen and smooth muscle cells, and to provide cross-sectional images of tissue microstructure with a resolution of 10 μm. As another example, Laser Speckle Imaging (LSI) may also be used to measure the stability of a plaque by employing a small-diameter, flexible optical bundle that is inserted into a intravascular catheter. The transmission of a laser signal by the optical bundle from an atherosclerotic plaque can then be recorded and evaluated (e.g., aortic plaque images are obtained by cyclically inserting the fiber bundle to mimic coronary motion) to measure plaque stability. As yet another example, magnetic resonance imaging (MRI) can be used to detect vulnerable plaques. Additionally, reflection spectroscopy can further be used to detect the vulnerable plaque by collecting reflectance spectra in the spectral region from 400 nm to 1700 nm and measuring lipid content in a plaque using the reflection spectra. The measurement of lipid content can then be compared with the thickness of the lipid core, as determined from histology, to further determine plaque stability.

With regard to the various therapeutic methods described herein, although certain embodiments of the methods disclosed herein only call for a qualitative assessment (e.g., the presence or absence of stable plaques in a subject), other embodiments of the methods call for a quantitative assessment (e.g., an amount of reduction of LDL-oxidation in a subject or an amount of vasodilation in a subject). Such quantitative assessments can be made, for example, using one of the above mentioned methods, as will be understood by those skilled in the art.

The skilled artisan will also understand that measuring a reduction in the amount of a certain feature (e.g., LDL-oxidation) or an improvement in a certain feature (e.g., vasodilation) in a subject is a statistical analysis. For example, a reduction in an amount of LDL-oxidation in a subject can be compared to a control level of LDL-oxidation, and an amount of LDL-oxidation of less than or equal to the control level can be indicative of a reduction in the amount of LDL-oxidation, as evidenced by a level of statistical significance. Statistical significance is often determined by comparing two or more populations, and determining a confidence interval and/or a p value. See, e.g., Dowdy and Wearden, Statistics for Research, John Wiley & Sons, New York, 1983, incorporated herein by reference in its entirety. Preferred confidence intervals of the present subject matter are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

The compounds of the present invention, including methylenedioxyphenyl ferulate (Formula (II)) and ferulylproline (Formula (XVI)) and derivatives thereof, are designed to be effective inhibitors of MPO. As such, it is believed that the presently-disclosed compounds will be useful as agents to reduce the amount of harmful oxidants or oxidized proteins. Along these lines, and as indicated above, it is further contemplated that the presently-disclosed compounds will be useful for treating cardiovascular disease in a variety of subjects where inhibition of MPO activity is indicated.

As used herein, the term “subject” includes both human and animal subjects. Thus, veterinary therapeutic uses are provided in accordance with the presently disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The embodiments of the presently-disclosed subject matter as set forth herein are subject to modifications, and other modified embodiments within the scope of the invention will be evident to those of ordinary skill in the art after a study of the information provided in this document. The information provided in this document, and particularly the specific details of the described exemplary embodiments, is provided primarily for clearness of understanding and no unnecessary limitations are to be understood therefrom.

Further, while the terms used in the application are believed to be well understood by one of ordinary skill in the art, definitions are set forth to facilitate explanation of the presently-disclosed subject matter. Unless defined otherwise, all technical and scientific terms used herein have the same meaning as commonly understood by one of ordinary skill in the art to which the present invention belongs. Although any methods, devices, and materials similar or equivalent to those described herein can be used in the practice or testing of the presently-disclosed subject matter, representative methods and materials have been described herein above.

Additionally, following long-standing patent law convention, the terms “a”, “an”, and “the” refer to “one or more” when used in this application, including the claims. Thus, for example, reference to “an inflammatory molecule” includes a plurality of such molecules, and so forth. Also, unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the present invention.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods.

### EXAMPLES

The following examples are provided which exemplify aspects of the preferred embodiments of the present invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Synthesis and Characterization of Methylenedioxyphenyl Ferulate

To synthesize methylenedioxyphenyl ferulate (Formula (II)), the chemicals and reagents for the synthesis procedure described below, including: ferulic acid, methylenedioxyphenol, dimethylaminopyridine (DMAP), triethylamine (TEA), and N-(3-Dimethylaminopropyl)-N′-ethylcarbodiimide hydrochloride (EDCl) were first purchased from Sigma-Aldrich Co. (St. Louis, Mo.).

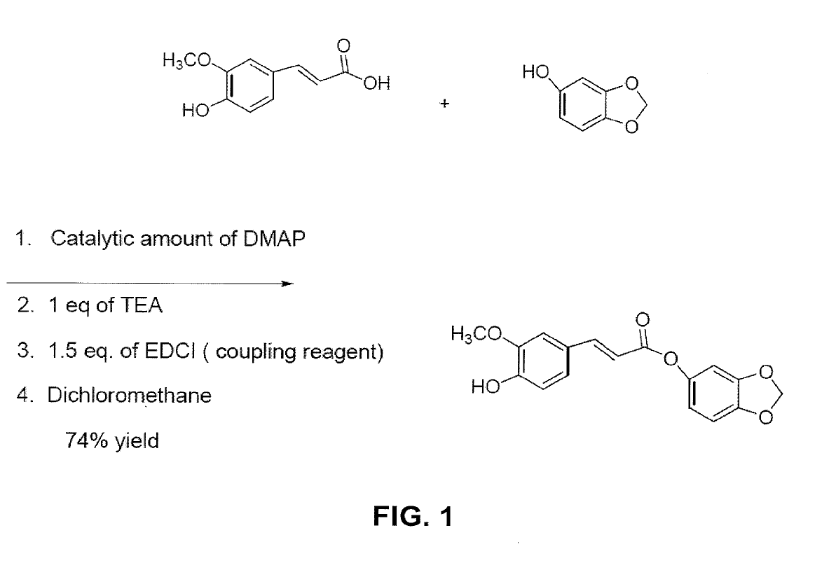

In the synthesis procedure, which is depicted in FIG. 1, the synthesis of methylenedioxyphenyl ferulate was accomplished by the use of a coupling reaction between ferulic acid and methylenedioxyphenol, with the entire reaction being carried out under a nitrogen atmosphere. To complete the reaction, ferulic acid, 0.194 g (1 millimole (mM)); methylenedioxyphenol, 0.138 g (1 mM); triethylamine 0.101 g, corresponding volume: 0.140 mL; and a catalytic amount (about 1 to 2 mg) of dimethylaminopyridine were initially combined in a 100 mL round bottom flask. The contents of the flask were then dissolved in dichloromethane (25 mL) and stirred well for 5 minutes at room temperature. Next, the coupling reagent EDCl, 0.287 g (1.5 mM) was added in portions over a 2 hour period while the contents of the flask were stirred simultaneously. Completion of the reaction was monitored using thin layer chromatography by comparing the disappearance of the starting materials and the formation of the new product.

After overnight stirring, dichloromethane was evaporated under reduced pressure using a rotary evaporator. The resultant crude product was purified using fluorescent preparative thin layer chromatography with a ratio of 50% ethylacetate:hexane. The appropriate required compound band was isolated, and the compound was extracted from it by continuously eluting with ethylacetate (300 mL). The solvent was then evaporated to dryness under a vacuum pump and a white solid was obtained in 74% yield. The compound was characterized by proton NMR spectrum and the typical proton chemical shift values were (CDCl₃): δ 7.79 (1H, doublet), 7.15-7.14 (1H, doublet), 7.126 (1H, singlet), 6.96-6.94 (1H, doublet), 6.81-6.79 (1H, doublet), 6.69-6.68 (1H, doublet), 6.62-6.62 (1H, doublet), 6.59-6.58 (1H, doublet), 6.47-6.43 (1H, doublet), 5.99 (2H, singlet), 3.95 (3H, singlet). The product was determined to have the following chemical structure and physical characteristics, confirming it as methylenedioxyphenyl ferulate (Formula (II)).

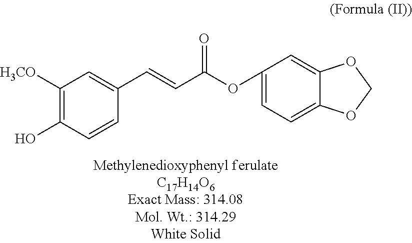

### Example 2

### Synthesis and Characterization of Ferulylproline

To synthesize ferulylproline (Formula (XVI)), the chemicals and reagents for the synthesis procedure described below, including: Ferulic acid, L-Proline methyl ester hydrochloride, Dimethylaminopyridine (DMAP), and Dicyclohexyl carbodiimide (DCC), were first purchased from Sigma-Aldrich Co. (St. Louis, Mo.). The synthesis of ferulylproline was accomplished through the use of a coupling reaction between ferulic acid and the methyl ester of proline followed by base-catalyzed hydrolysis of the ester to regenerate the free carboxylic acid. The entire reaction was carried out under a nitrogen atmosphere.

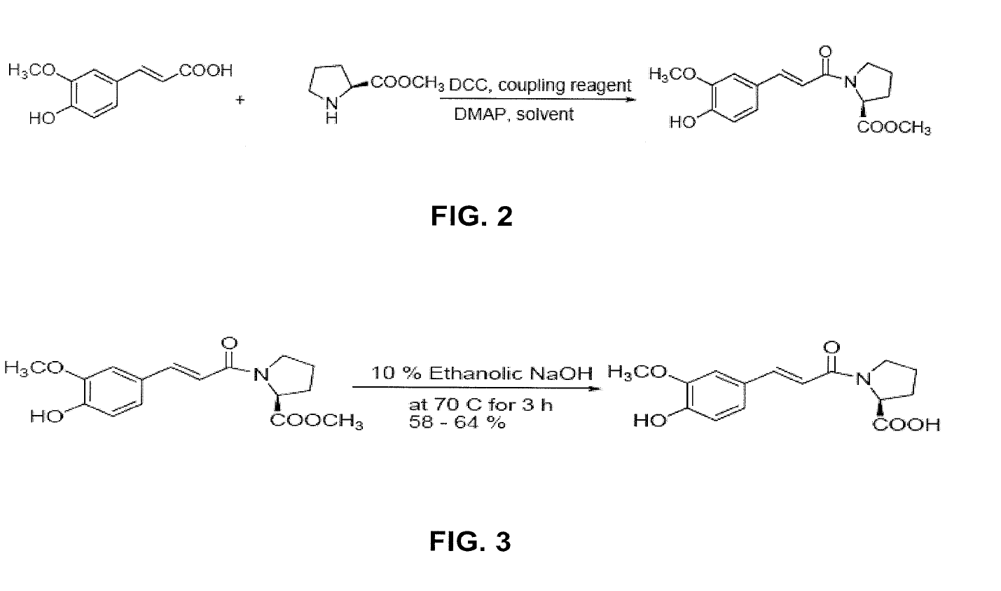

In the first step of the reaction, which is depicted in FIG. 2, ferulic acid, 0.194 g (1 mM); proline ester, 0.165 g (1 mM); and DMAP 0.101 g, were combined in a 100 mL round bottom flask. The contents of the flask were dissolved in dichloromethane (25 mL) and stirred well for 5 minutes at room temperature. Completion of the reaction was then monitored using thin layer chromatography by comparing the disappearance of the starting materials and the formation of the new product.

After overnight stirring, dichloromethane was then evaporated under reduced pressure using a rotary evaporator. The resultant crude product, namely ferulylproline methyl ester, was subsequently purified using column chromatography with a ratio of 150% ethylacetate:hexane. The appropriate required compound band was isolated, and used directly for next step. The total yield of this reaction was 91%.

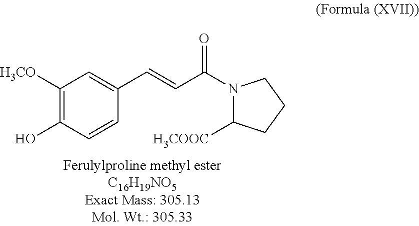

In the second step of the synthesis procedure, shown in FIG. 3, the free acid was obtained by treating the esterified compound with 10% ethanolic sodium hydroxide and stirring for 3 hours at 70° C. The solvent ethanol was evaporated and the crude compound was acidified with cold, dilute hydrochloric acid. Free carboxylic acid was purified on silica gel column chromatography. The final acid was obtained as a pale yellow low melting solid in 58 to 64% chemical yield. The compound was characterized by proton NMR and chemical shift values are assigned as follows: (CDCl₃): δ 7.74-7.71 (1H, doublet), 6.99-6.98 (1H, doublet), 6.92-6.90 (1H, doublet), 6.54-6.50 (1H, doublet), 4.72-4.70 (2H, triplet), 3.92 (3H, singlet), 3.75-3.72 (2H, triplet), 3.66-3.63 (2H, quartet), 2.57-2.54 (1H, multiplet), 2.07-2.04 (3H, multiplet). The product was determined to have the following chemical structure and physical characteristics, confirming it as ferulylproline (Formula (XVI)).

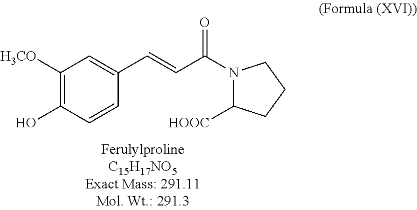

### Example 3

### Inhibition of Myeloperoxidase (MPO) Activity

To determine if the compounds of the present invention inhibit myeloperoxidase (MPO) activity at various concentrations, tetramethyl benzidine (TMB) was used as a substrate in an MPO assay as it was more sensitive than guaiacol and the color was more stable. Typically, the reaction mixture (200 μl) contained 20 mU human MPO (Sigma-Aldrich, St. Louis, Mo.), 400 nmol H₂O₂, 1.6 μmol of TMB, and varying concentrations of methylenedioxyphenyl ferulate (Formula (II)) or ferulylproline (Formula (XVI)).

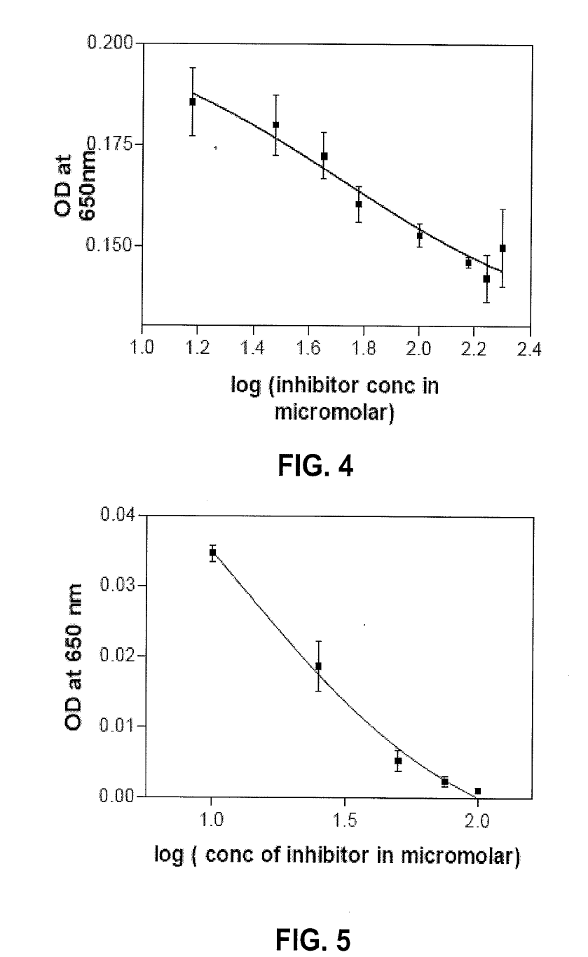

The reaction was performed in a volume of 200 μl with 50 mM sodium acetate buffer at pH 5.6. The reaction was initiated by adding MPO and the optical density of the product formed was then read at 650 nm in a microplate reader at various time points. The results demonstrated that both of the compounds, methylenedioxyphenyl ferulate and ferulylproline, significantly inhibited MPO, as shown in FIGS. 4 and 5. Furthermore, the inhibitory constants (Ki) calculated from the studies revealed Ki values that were physiologic and could be achieved through oral dosing (Table 2).

TABLE 2
Inhibitory values of MPO for methylenedioxyphenyl ferulate and
ferulylproline.

| Compound | Ki |
|---|---|
| methylenedioxyphenyl ferulate | 8.5 μM |
| ferulylproline | 4 μM |

### Example 4

### Inhibition of Low-Density Lipoprotein Oxidation

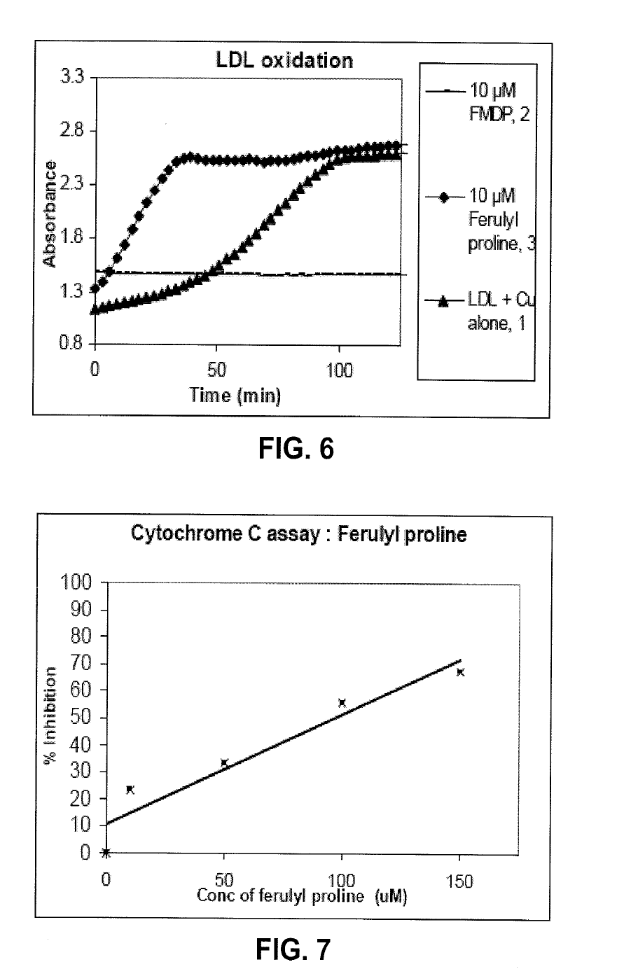

To determine the effect of the presently-disclosed compounds on low-density lipoprotein (LDL) oxidation, standard LDL oxidation reactions were carried out in the presence of methylenedioxyphenyl ferulate (FMDP; Formula (II)) and ferulylproline (Formula (XVI). Briefly, the in vitro oxidation reaction of LDL was carried out at room temperature in potassium phosphate buffer, pH 7.4. The reaction mixture contained 100 μg LDL protein and 5 μM Cu (II) with a final volume of 1 mL. The oxidation of LDL was followed by measuring the formation of a conjugated diene at 234 nm for a total of 300 min using a spectrophotometer. The time course of Cu (II) induced LDL oxidation was then performed. At concentrations of 10 and 25 μM of methylenedioxyphenyl ferulate, complete inhibition of LDL oxidation was observed (FIG. 6). Under the same conditions, ferulylproline acted as a pro-oxidant reducing the lag time (FIG. 6). The increase in the oxidation of LDL in presence of 10 μM and 25 μM ferulylproline was attributed to the formation of Cu (II)-ferulylproline complex which would act as a pro-oxidant of LDL. Oxidation of LDL lipid by Cu (II) mediated oxidation both in the absence of any inhibitor and in the presence of 10 μM and 25 μM of methylenedioxyphenyl ferulate and ferulylproline was performed by monitoring the oxidation for 200 minutes.

### Example 5

### Inhibition of Cytochrome C Oxidation

To determine if the compounds of the present invention were capable of effectively inhibiting the reduction of cytochrome c, cytochrome c was reduced by a superoxide radical generated by xanthine/xanthine oxidase and the product of cytochrome reduction was observed at 549 nm in a spectrophotometer. The reaction mixture contained 250 μL of 5 mM hypoxanthine (1.25 mM final concentration), 100 μL of cytochrome c in buffer, 10 μL of xanthine oxidase enzyme and different concentrations of inhibitor. The final volume was made up to 1 mL with phosphate buffer. The oxidation was monitored by scanning wavelengths between 500 nm to 600 nm with five measurements. The cycle scanning time was 30 seconds. The results revealed that ferulylproline inhibited the reaction (FIG. 7), while methylenedioxyphenyl ferulate did not cause significant inhibition indicating specificity of the described compounds toward inhibition of MPO.

### Example 6

### Inhibition of Chlorotaurine Formation

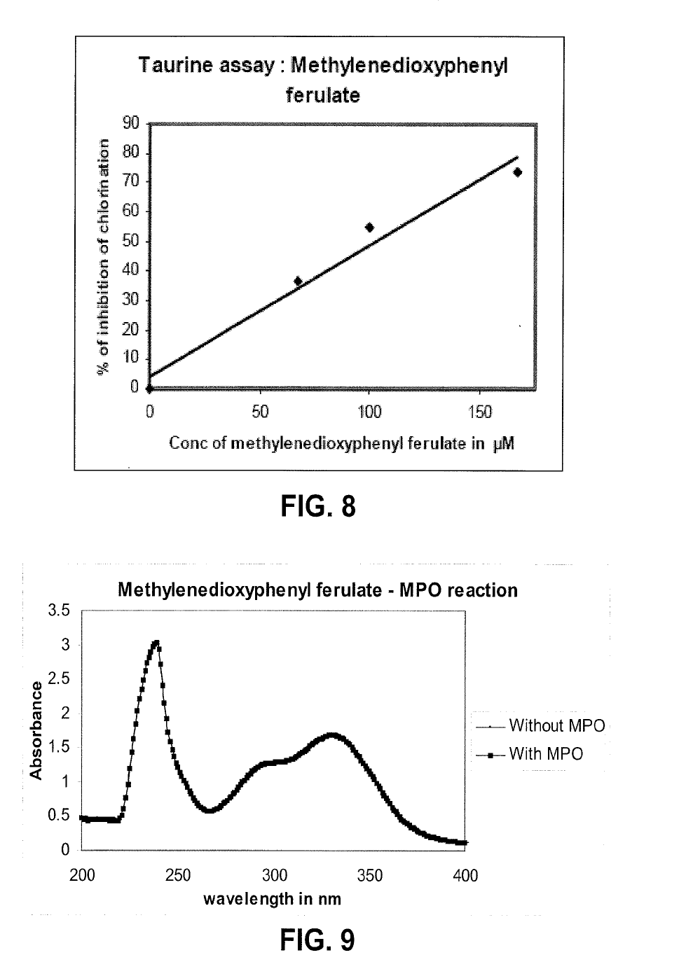

Chlorotaurine is formed by the oxidation of taurine by the chlorinating oxidant hypochlorous acid (HOCl), which is formed by MPO-mediated 2-electron oxidation of chloride by H₂O₂. To determine if the compounds of the present invention were capable of inhibiting the formation of chlorotaurine, a reaction mixture was produced that contained 50 uL of 150 mM taurine, various concentrations of methylenedioxyphenyl ferulate (Formula (II)) or ferulylproline (Formula (XVI)), and 250 μL of hypochlorous acid in sodium phosphate buffer. The mixture was incubated at 37° C. for 10 min followed by addition of 60 μL of 40 mM thionitrobenzoic acid (TNB). The whole solution was made up to 1.5 mL and absorption was measured at 412 nm using a UV-Visible spectrophotometer. The results indicated that while methylenedioxyphenyl ferulate inhibited the reaction (FIG. 8), ferulylproline did not cause any inhibition. Furthermore, although both of these compounds react differently toward chlorotaurine oxidation, they seem to show specificity toward MPO inhibition.

### Example 7

### Analysis of Myeloperoxidase Inhibition

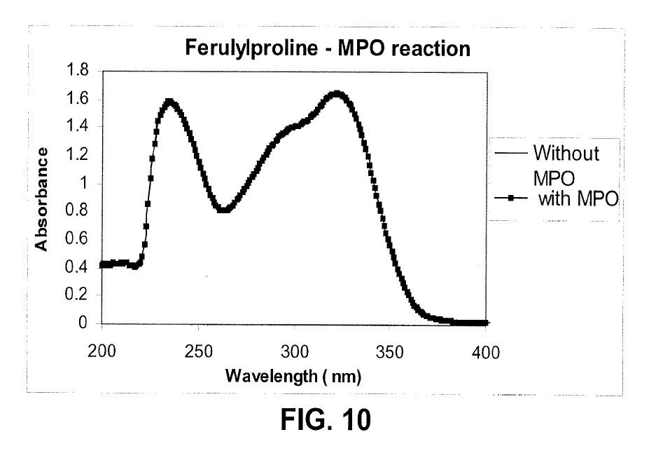

It is known that MPO acts on a variety of substrates and, as such, it was thought that it may be theoretically possible that methylenedioxyphenyl ferulate (Formula (II)) and ferulylproline (Formula (XVI)) could be acting as MPO substrates and competitively masking the formation of products from the added substrates in the above-described experiments. However, measurement of the UV-Visible spectrum of methylenedioxyphenyl ferulate and ferulylproline between 200 to 700 nm with and without MPO failed to show any product (FIGS. 9 and 10). In these experiments, a reaction mixture was produced that contained 720 μL of sodium acetate buffer, 100 μL of 100 mM H₂O₂, 100 μL of MPO enzyme with added compounds of the present invention, and a total volume made up to 1 mL. Upon analysis of this reaction mixture, however, there was no indication of any colored product even when the reaction mixture contained only the inhibitor and not the MPO substrate. To strengthen this finding, thin layer chromatography (TLC) was run with the MPO assay solution and inhibitor (50% ethylacetate:hexane) and spot identification was done in an iodine chamber. The TLC experiment did not show any product formation during the MPO reaction indicating methylenedioxyphenyl ferulate and ferulylproline do not act as an MPO substrate, but should instead be characterized as inhibitors of MPO.

Throughout this document, various references are mentioned. All such references are incorporated herein by reference. It will also be understood that various details of the present invention can be changed without departing from the scope of the subject matter disclosed herein. Furthermore, the foregoing description is for the purpose of illustration only, and not for the purpose of limitation.

1. A compound having the Formula (I): 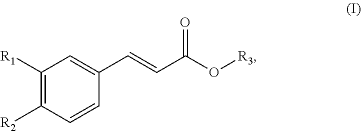
wherein:
R₁ is selected from the group consisting of OCH₃, OH, and OCH₂CH₃;
R₂ is selected from the group consisting of OH and OCH₃; and
R₃ is selected from the group consisting of: 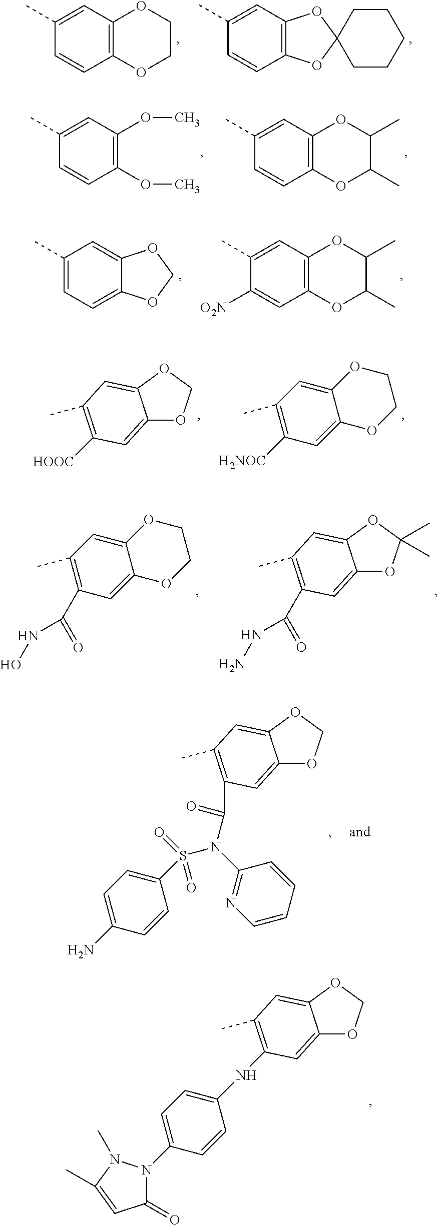
or a pharmaceutically-acceptable salt or solvate thereof.

2. The compound of claim 1, wherein the compound has a formula selected from the group consisting of Formula (II): 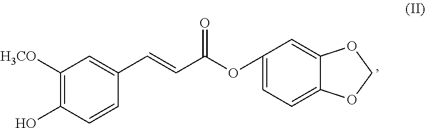
Formula (III): 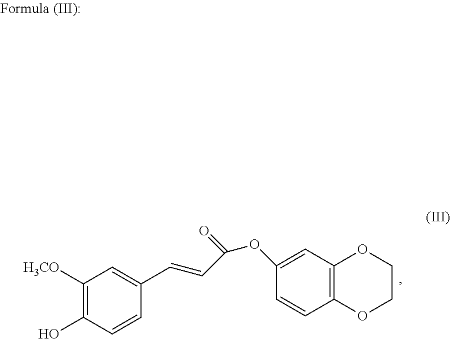
Formula (IV): 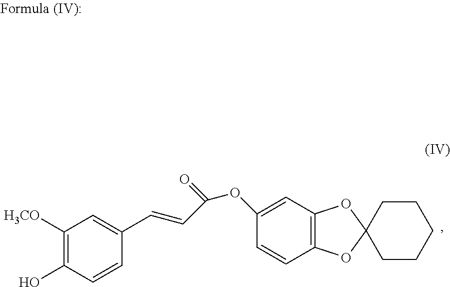
Formula (V): 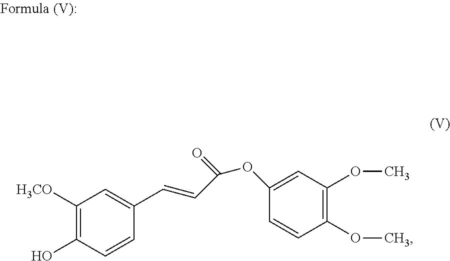
Formula (VI): 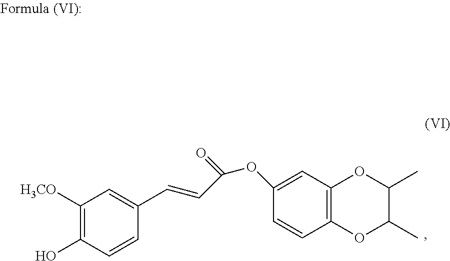
Formula (VII): 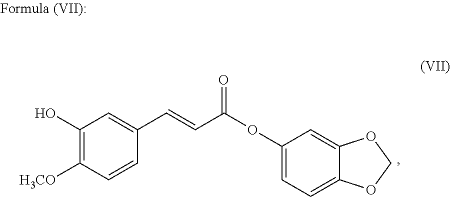
Formula (VIII): 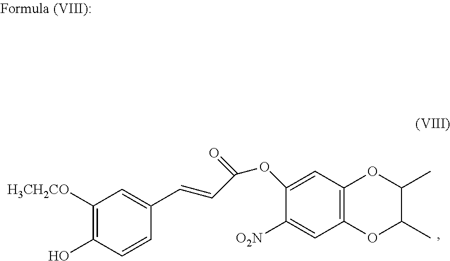
Formula (IX): 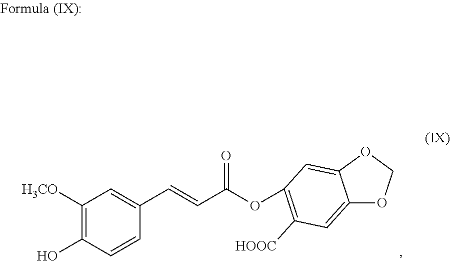
Formula (X): 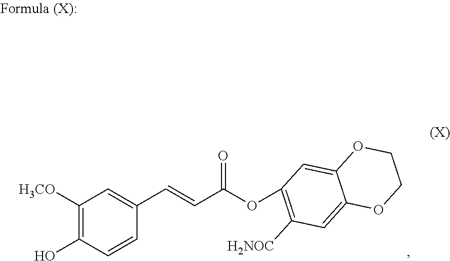
Formula (XI): 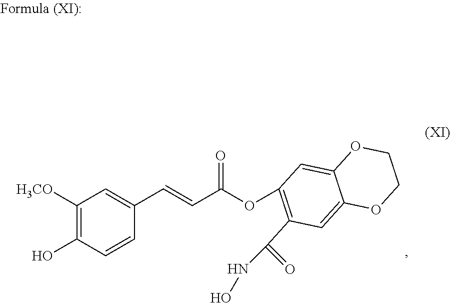
Formula (XII): 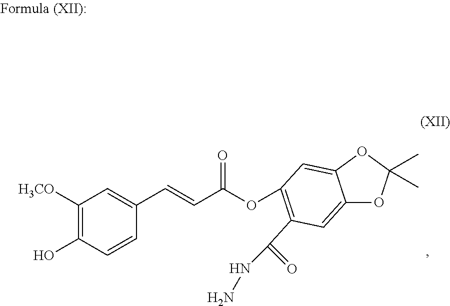
Formula (XIII): 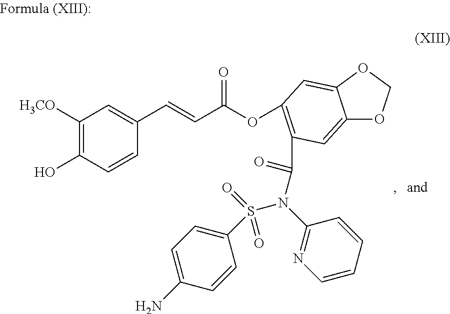
Formula (XIV):

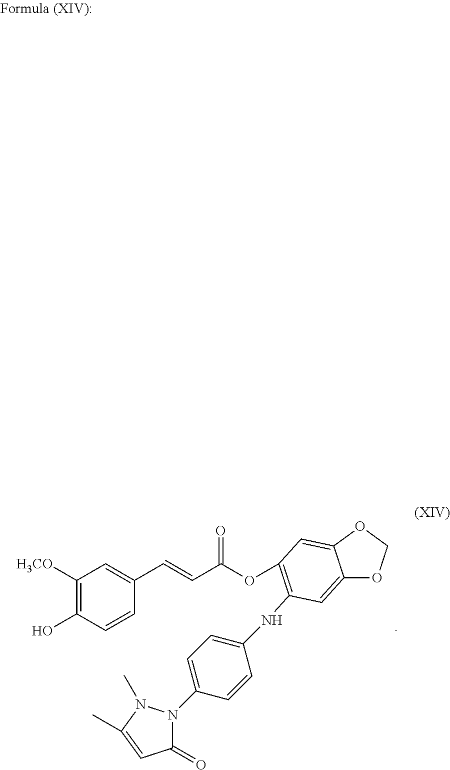

3-14. (canceled)

15. A pharmaceutical composition, comprising a compound of claim 1 and a pharmaceutically-acceptable vehicle, carrier, or excipient.

16. A compound having the Formula (XV): 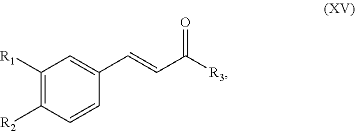
wherein:
R₁ is selected from the group consisting of OCH₃, OCH₂CH₃, and CONHNH₂;
R₂ is selected from the group consisting of OH and NH₂; and
R₃ is selected from the group consisting of: 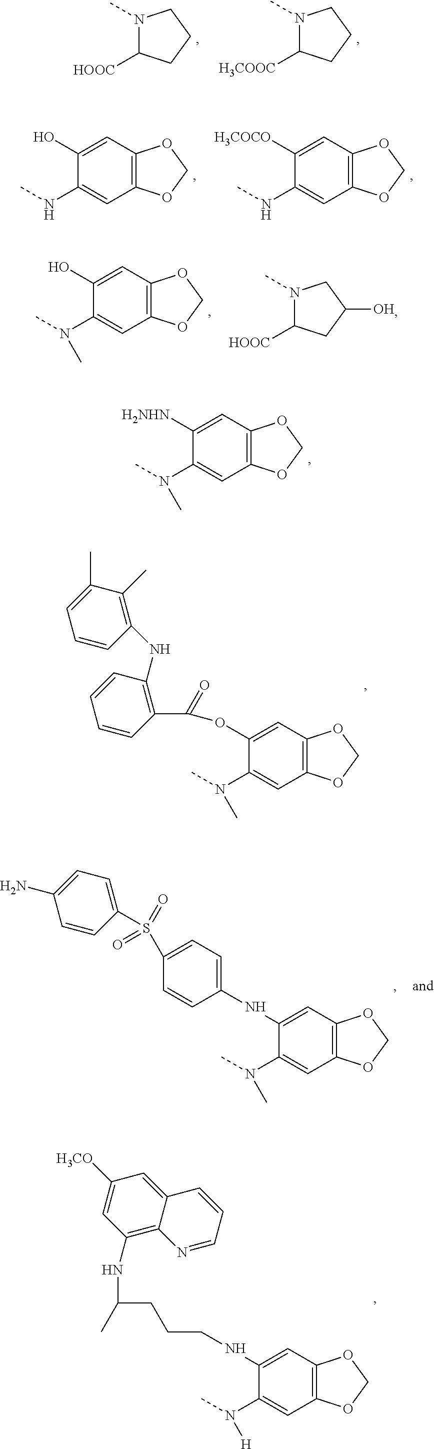
or pharmaceutically-acceptable salt or solvate thereof.

17. The compound of claim 16, wherein the compound has a formula selected from the group consisting of Formula (XVI): 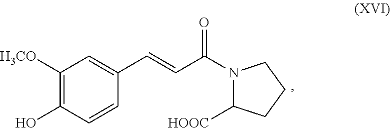
Formula (XVII): 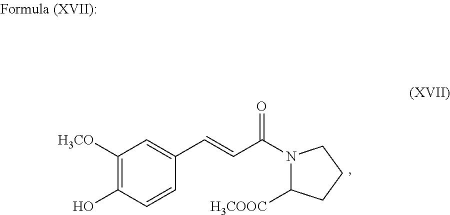
Formula (XVIII): 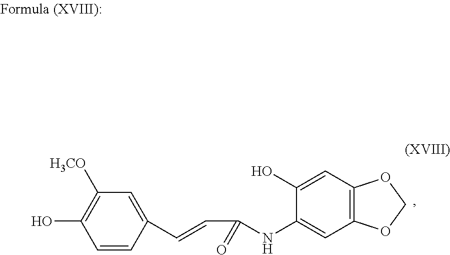
Formula (XIX): 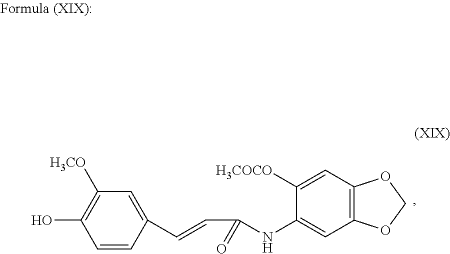
Formula (XX): 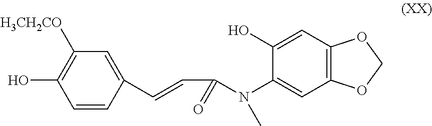
Formula (XXI): 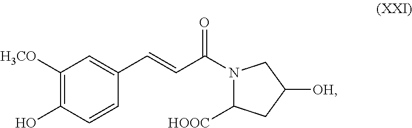
Formula (XXII): 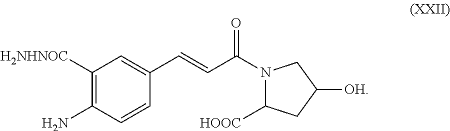
Formula (XXIII): 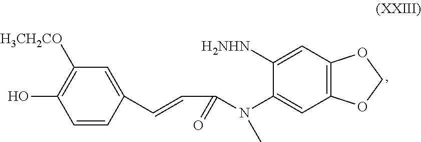
Formula (XXIV): 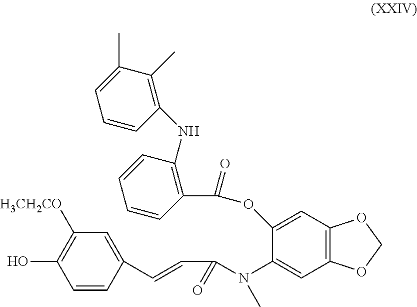
Formula (XXV): 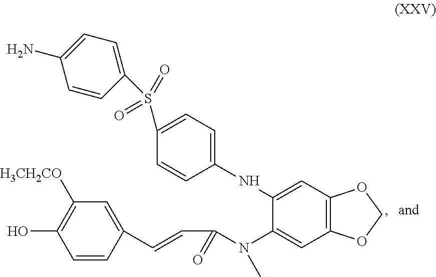
Formula (XXVI):

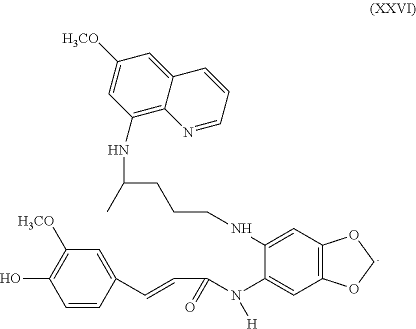

18-27. (canceled)

28. A pharmaceutical composition, comprising a compound of claim 16 and a pharmaceutically-acceptable vehicle, carrier, or excipient.

29. A compound having the Formula (XXVII): 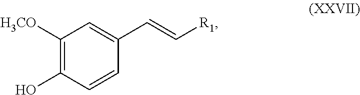
wherein R₁ is selected from the group consisting of: 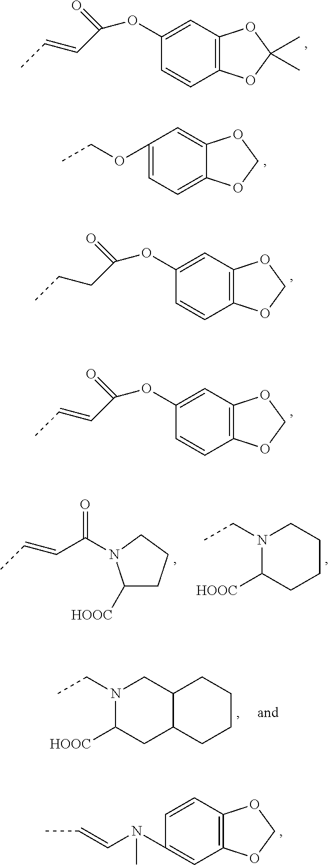
or a pharmaceutically acceptable salt or solvate thereof.

30. The compound of claim 29, wherein the compound has a formula selected from the group consisting of Formula (XXVIII): 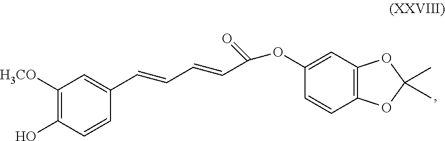
Formula (XXIX): 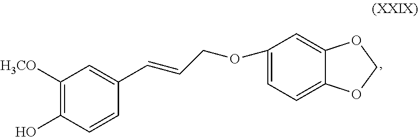
Formula (XXX): 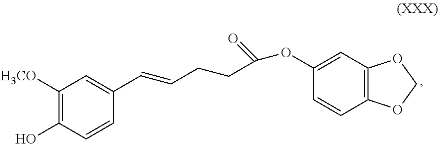
Formula (XXXI): 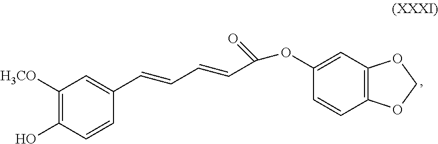
Formula (XXXII): 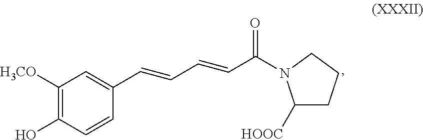
Formula (XXXIII): 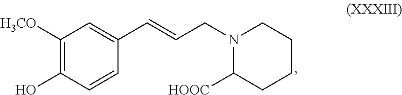
Formula (XXXIV): 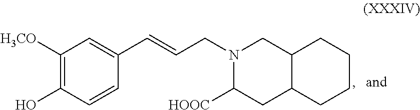
Formula (XXXV):

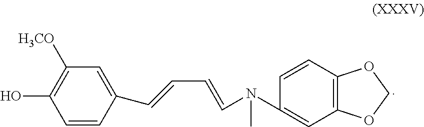

31-37. (canceled)

38. A compound having a formula selected from the group consisting of the following Formulas (II) and (XVI), or pharmaceutically-acceptable salts or solvates thereof:

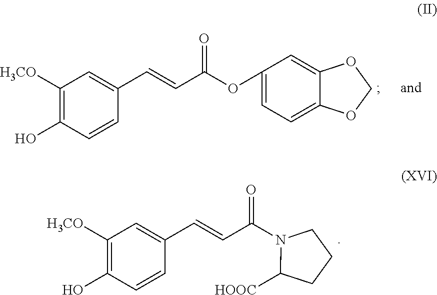

39. A method of reducing myeloperoxidase enzyme activity, comprising contacting a myeloperoxidase enzyme with an effective amount of a compound selected from the group consisting of the following Formulas (I), (XV) and (XXVII), or pharmaceutically-acceptable salts or solvates thereof: 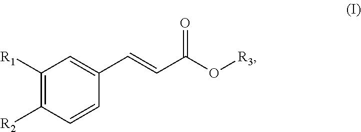
wherein:
R₁ is selected from the group consisting of OCH₃, OH, and OCH₂CH₃;
R₂ is selected from the group consisting of OH and OCH₃; and
R₃ is selected from the group consisting of: 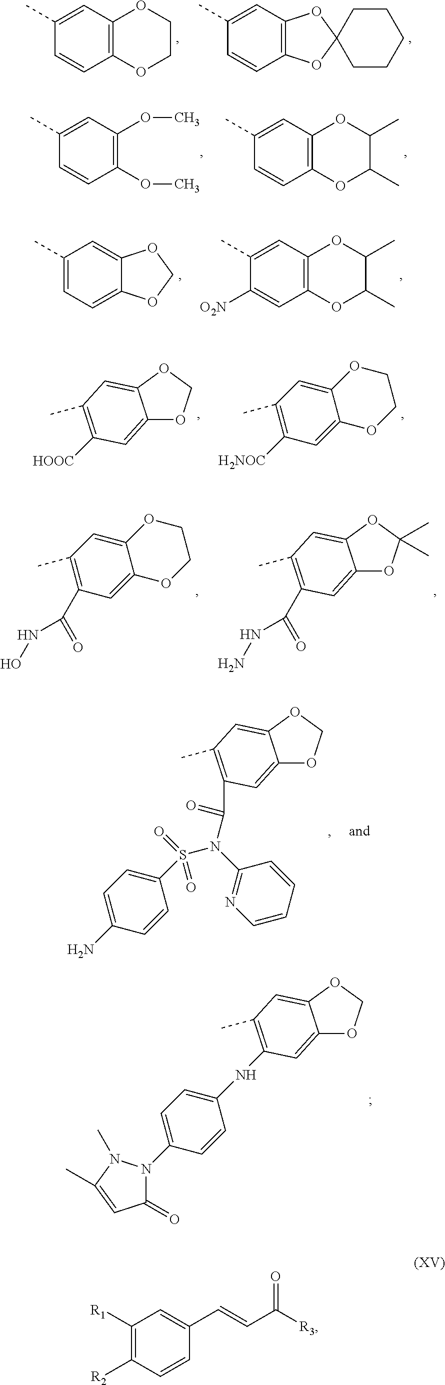
wherein:
R₁ is selected from the group consisting of OCH₃, OCH₂CH₃, and CONHNH₂;
R₂ is selected from the group consisting of OH and NH₂; and
R₃ is selected from the group consisting of: 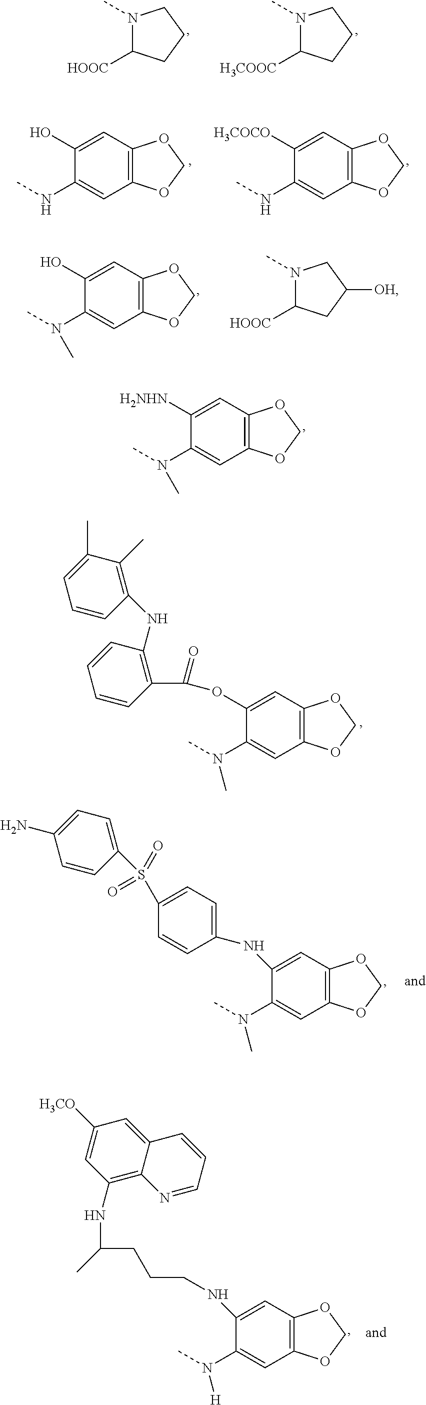 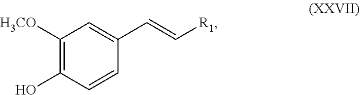
wherein R₁ is selected from the group consisting of:

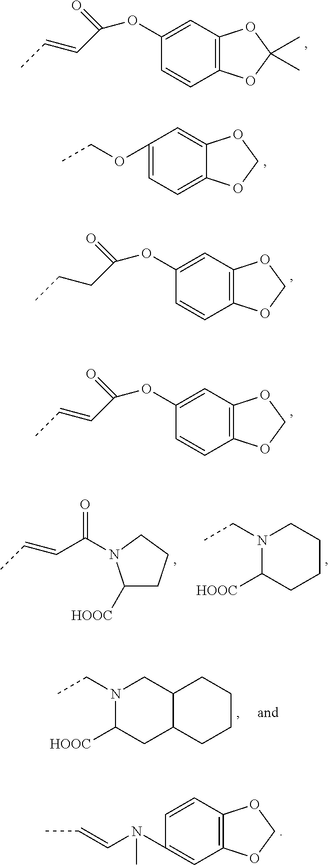

40. A method for treating a cardiovascular disease, comprising administering to a subject in need thereof an effective amount of a compound selected from the group consisting of the following Formulas (I), (XV) and (XXVII), or pharmaceutically-acceptable salts or solvates thereof: 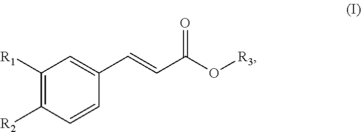
wherein:
R₁ is selected from the group consisting of OCH₃, OH, and OCH₂CH₃;
R₂ is selected from the group consisting of OH and OCH₃; and
R₃ is selected from the group consisting of: 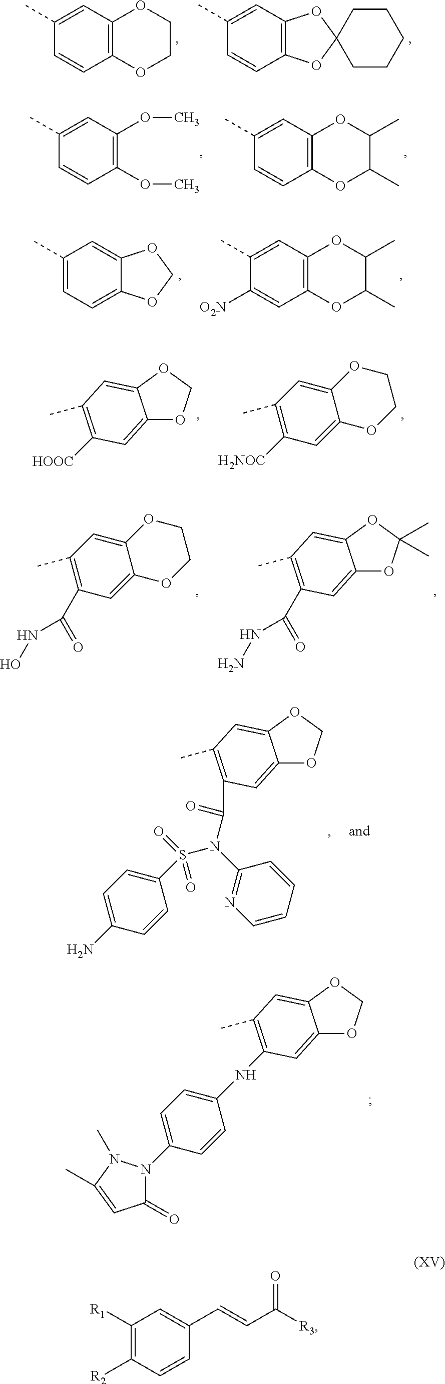
wherein:
R₁ is selected from the group consisting of OCH₃, OCH₂CH₃, and CONHNH₂;
R₂ is selected from the group consisting of OH and NH₂; and
R₃ is selected from the group consisting of: 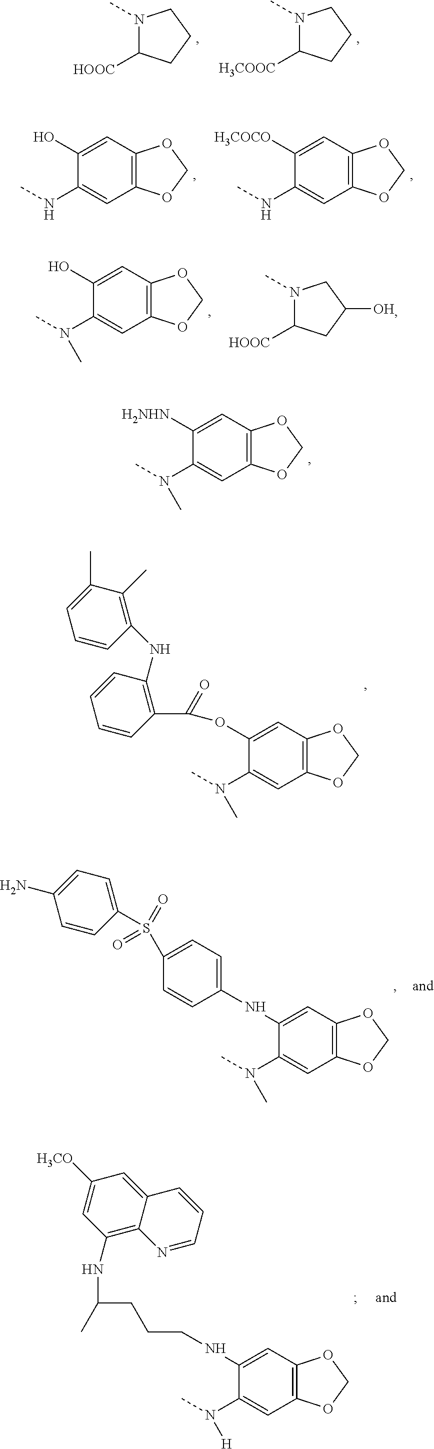 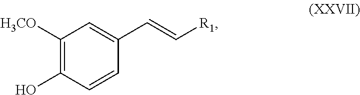
wherein R₁ is selected from the group consisting of:

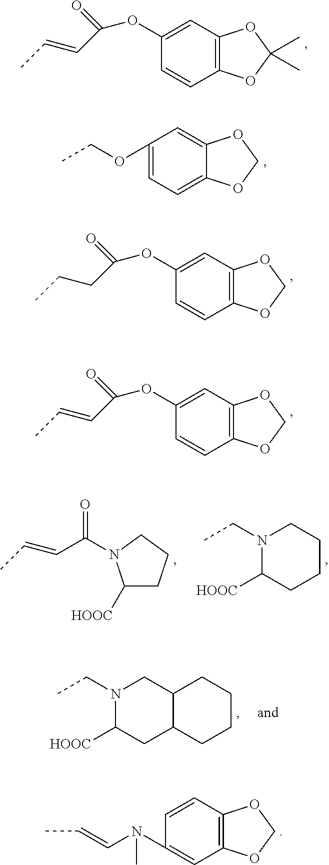

41. The method of claim 40, wherein the cardiovascular disease is selected from the group consisting of hypertension, atherosclerosis, coronary heart disease, angina, stroke, and myocardial infarction.

42. The method of claim 40, further comprising administering to the subject an effective amount of an angiotensin-converting enzyme inhibitor, an angiotensin II receptor blocker, a statin, an anti-inflammatory agent, an agent that inhibits the absorption of fatty acids, an anti-platelet agent, an anti-coagulant, or combinations thereof.

43. A method for reducing low-density lipoprotein oxidation, comprising administering to a subject in need thereof an effective amount of a compound selected from the group consisting of the following Formulas (I), (XV) and (XXVII), or pharmaceutically-acceptable salts or solvates thereof:
(I) 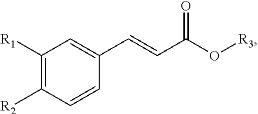
wherein:
R₁ is selected from the group consisting of OCH₃, OH, and OCH₂CH₃;
R₂ is selected from the group consisting of OH and OCH₃; and
R₃ is selected from the group consisting of: 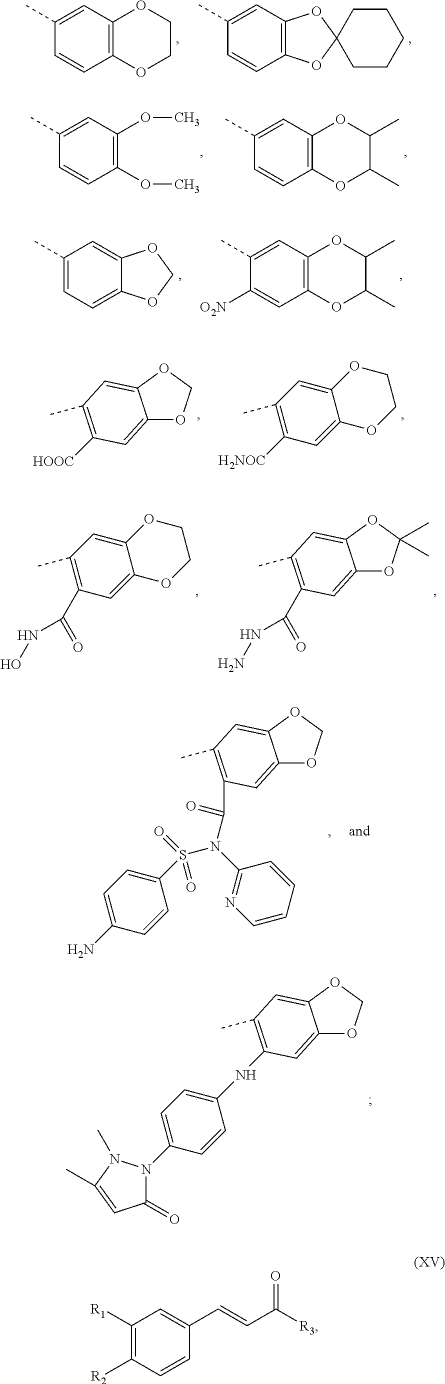
wherein:
R₁ is selected from the group consisting of OCH₃, OCH₂CH₃, and CONHNH₂;
R₂ is selected from the group consisting of OH and NH₂; and
R₃ is selected from the group consisting of: 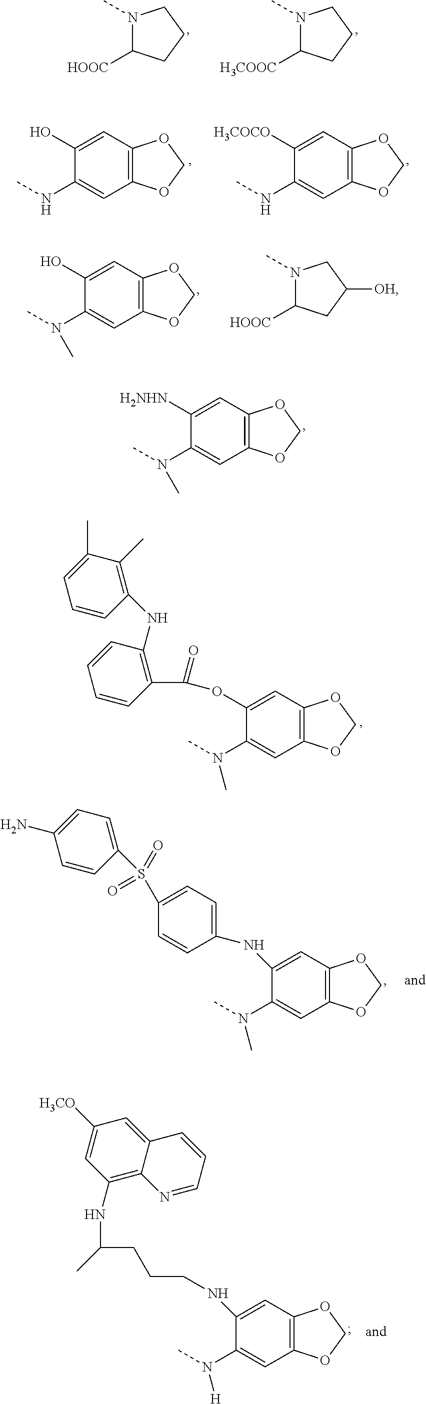 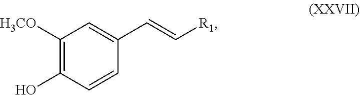
wherein R₁ is selected from the group consisting of:

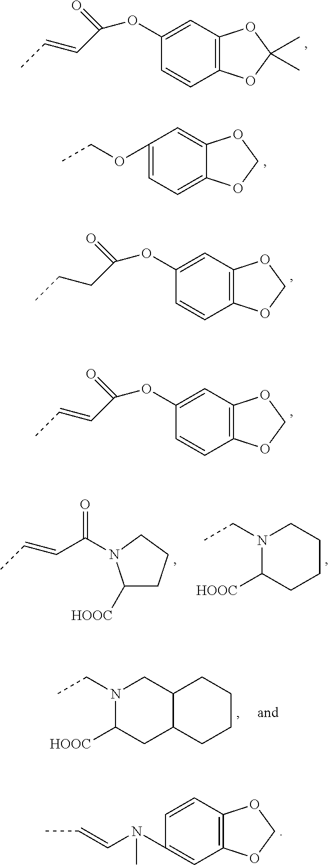

44. A method for increasing vasodilation, comprising administering to a subject in need thereof an effective amount of a compound selected from the group consisting of the following Formulas (I), (XV) and (XXVII), or pharmaceutically-acceptable salts or solvates thereof: 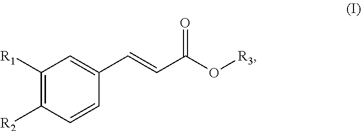
wherein:
R₁ is selected from the group consisting of OCH₃, OH, and OCH₂CH₃;
R₂ is selected from the group consisting of OH and OCH₃; and
R₃ is selected from the group consisting of: 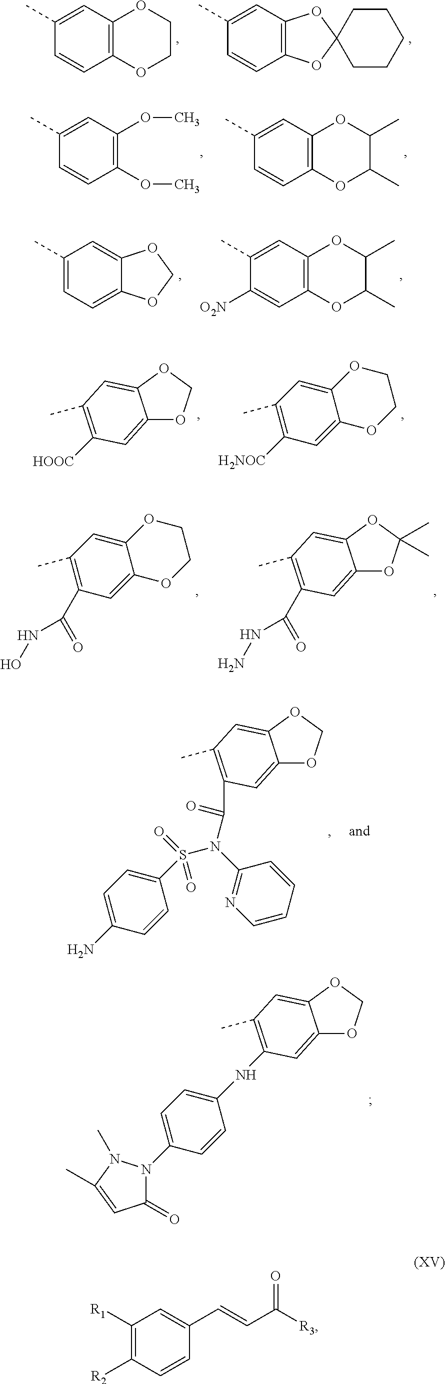
wherein:
R₁ is selected from the group consisting of OCH₃, OCH₂CH₃, and CONHNH₂;
R₂ is selected from the group consisting of OH and NH₂; and
R₃ is selected from the group consisting of: 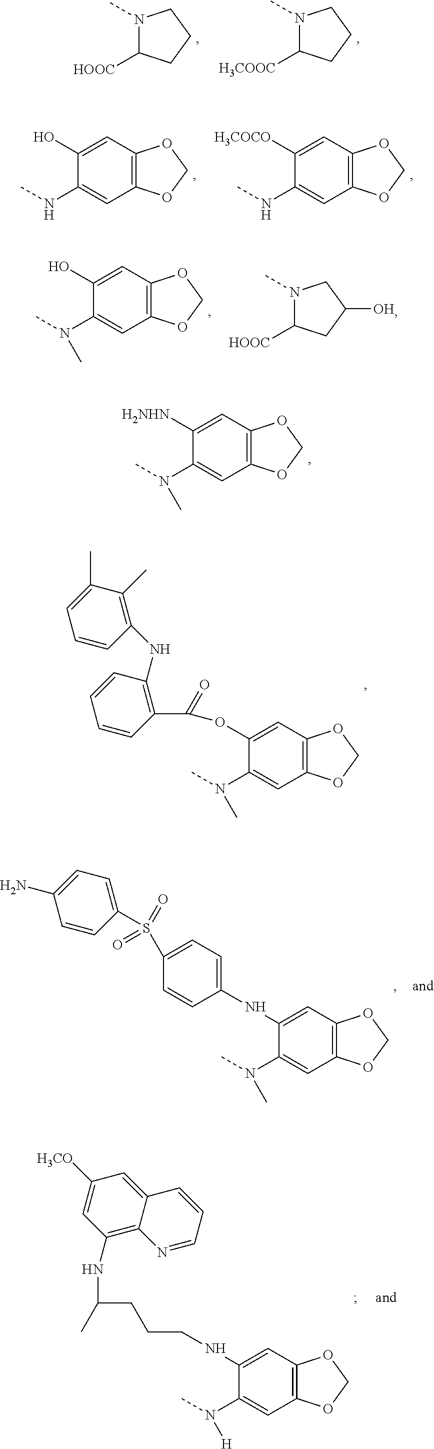 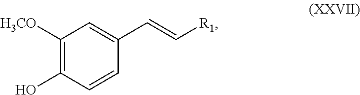
wherein R₁ is selected from the group consisting of:

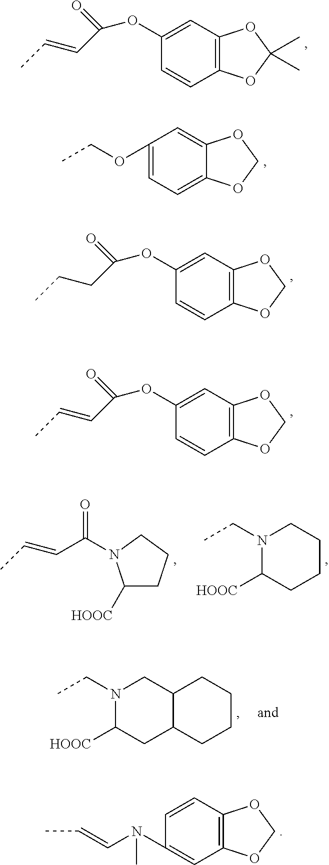

45. A method for reducing plaque destabilization, comprising administering to a subject in need thereof an effective amount of a compound selected from the group consisting of the following Formulas (I), (XV) and (XXVII), or pharmaceutically-acceptable salts or solvates thereof: 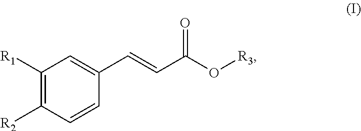
wherein:
R₁ is selected from the group consisting of OCH₃, OH, and OCH₂CH₃;
R₂ is selected from the group consisting of OH and OCH₃; and
R₃ is selected from the group consisting of 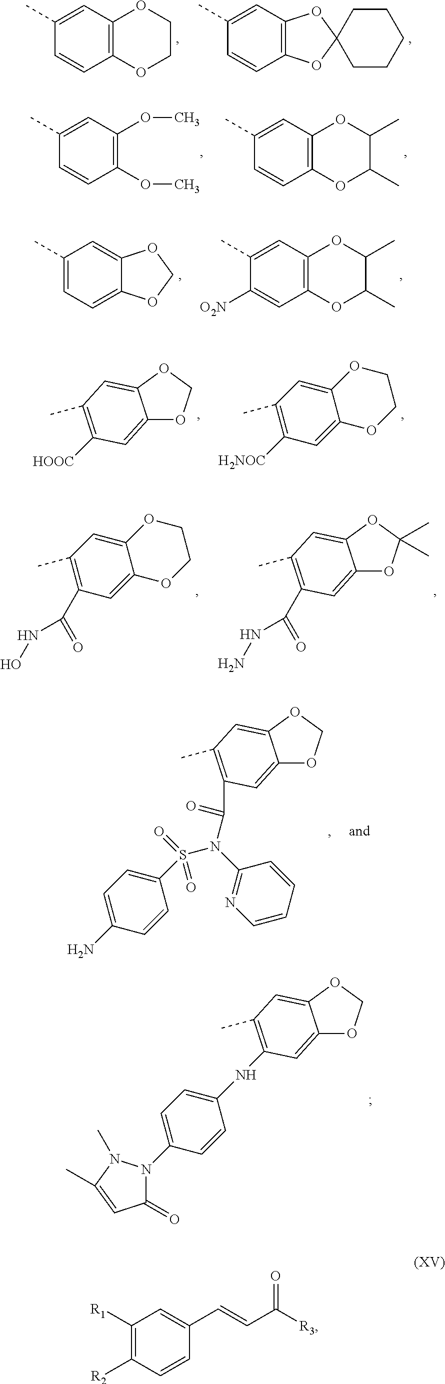
wherein:
R₁ is selected from the group consisting of OCH₃, OCH₂CH₃, and CONHNH₂;
R₂ is selected from the group consisting of OH and NH₂; and
R₃ is selected from the group consisting of: 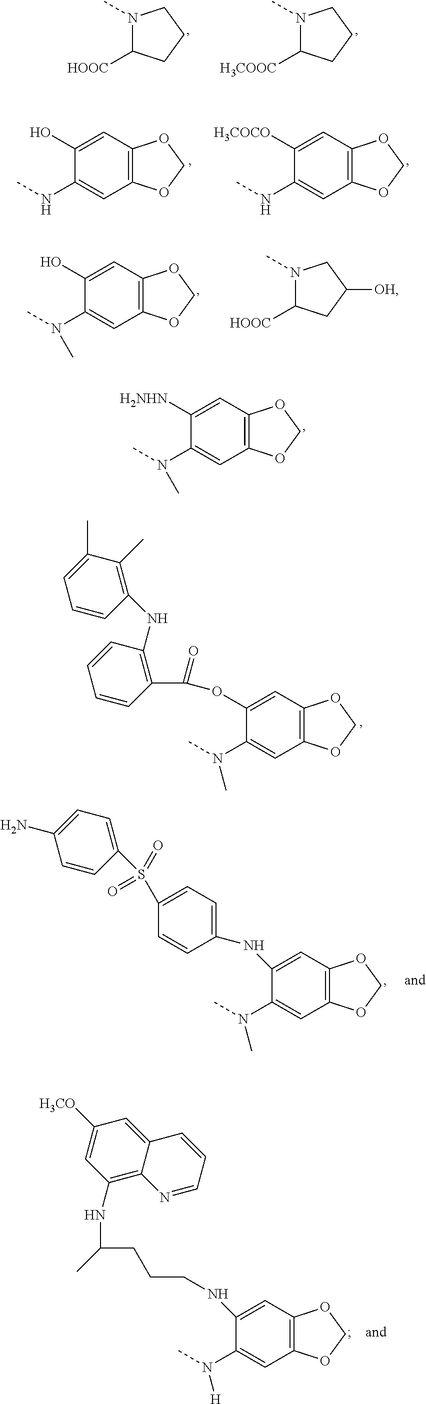 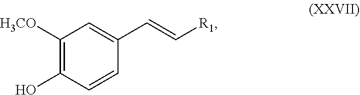
wherein R₁ is selected from the group consisting of: [chemical structure]
